(19) United States
(12) Patent Application Publication
(10) Pub. No.: US 2021/0284618 A1
(43) Pub. Date: Sep. 16, 2021

(54) FURANOCHALCONES AS INHIBITORS OF CYP1A1, CYP1A2 AND CYP1B1 FOR CANCER CHEMOPREVENTION

(71) Applicants: Council of Scientific & Industrial Research, New Delhi (IN); De Montfort University, Leicester (GB)

(72) Inventors: Sandip Bibishan Bharate, Jammu (IN); Rajni Sharma, Jammu (IN); Prashant Joshi, Jammu (IN); Ram Vishwakarma, Jammu (IN); Bhabatosh Chaudhuri, Leicester (GB)

(21) Appl. No.: 16/325,002

(22) PCT Filed: Aug. 11, 2017
(86) PCT No.: PCT/IN2017/050340

§ 371 Date: Feb. 12, 2019

(30) Foreign Application Priority Data
Aug. 12, 2016 (IN) 201611027579

(51) Int. Cl. C07D 307/86; C07D 493/04; C07D 409/06; C07D 405/06

(52) U.S. Cl. CPC C07D 307/86; C07D 405/06; C07D 409/06; C07D 493/04

(57) ABSTRACT

The present invention relates to the furanochalcone class of compounds of general formula A. The present invention particularly relates to the synthesis of furanochalcones and their CYP1A1, CYP1A2 and CYP1B1 inhibitory activity. In addition, the invention relates to the prevention or treatment of cancer caused by polyaromatic hydrocarbons (PAHs), 4-nitroquinoline-1-oxide, and N-nitroso-N-methylurea, heterocyclic amines, estrogen and 17β-estradiol, resulting from the inhibition of CYP1A1, CYP1A2 and CYP1B1 enzymes.

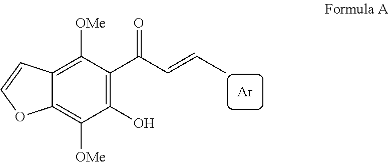

### FIELD OF THE INVENTION

The present invention relates to furanochalcone class of compounds as potent inhibitors of CYP1A1, CYP1A2 and CYP1B1 enzymes. The present invention also relates to a process for preparation of furanochalcones. More particularly, the present invention relates to the methods for the prevention or treatment of cancer, including those caused by carcinogenic harmful chemicals like benzo[a]pyrene (BaP) and 7,12-dimethylbenz[a]anthracene (DMBA). Compounds of the invention can be used as cancer chemopreventive agents.

### BACKGROUND OF THE INVENTION

Cancer is a group of diseases involving abnormal cell growth and with further potential to invade or spread to other parts of the body. The onset of cancer can be triggered by multiple factors alone or in combination including genetic, cellular physiological factors or external factors like physical carcinogens like ultraviolet and ionizing radiation, chemical carcinogens such as asbestos, arsenic, benzo[a]pyrene, DMBA or biological carcinogens like infections from certain viruses, bacteria or parasites (Badal, S. et. al. Enzymology. 2013, 1, 8). Various chemo-preventive measures could be adopted to protect healthy tissue by preventing, reversing or inhibiting the process of carcinogenesis that include cytochrome P450 (CYP450) enzyme inhibition (Schwartz, G. et. al. J. Clin. Oncol. 2005, 23, 9408; Stoner, G. et. al. Environ. Health Perspect. 1997, 105, 945).

Cytochrome P450 (CYP) enzymes are a large family of detoxification enzymes present in the human body. The human cytochrome P450-1 (CYP1) family consists of three members namely CYP1A1, CYP1A2 and CYP1B1. The expression of all three isozymes, CYP1A1, CYP1A2 and CYP1B1 is induced by poly-aromatic hydrocarbons (PAHs) which are found mainly in cigarette smoke, high-boiling fraction of crude oil, charred meat and vegetables. PAHs like 2,3,7,8-tetrachlorodibenzo-p-dioxin (TCDD), benzo(a)pyrene (BaP) and 7,12-dimethylbenz(a)anthracene (DMBA) have the ability to bind to aromatic hydrocarbon receptors (AhR) as ligands. The ligand-bound activated AhR performs the role of a transcription factor, and is responsible for the induction of CYP1 genes. This induction leads to increased levels of CYP1 enzymes (Wei, Q. et. al. Cancer Res. 1996, 56, 3975; Buterin, F. et. al. Cancer Res. 2000, 60, 1849). The PAHs also act as ideal substrates for CYP1 enzymes which efficiently hydroxylate the PAHs leading to the formation of carcinogenic entities from pro-carcinogenic molecules. The PAHs appear to play a dominant role in the CYP1-mediated positive feedback mechanism that underlies the formation of carcinogenic substances capable of intercalating DNA. Hydroxylated PAHs are carcinogenic since they have great propensity to intercalate with double-stranded DNA and then cause breaks in the double-stranded DNA. Hence, all PAHs in general have tumor promoting properties. Besides PAHs and its derivatives, CYP1 enzymes metabolize other xenobiotic compounds such as nitrogenous heterocycles, caffeine, aromatic amines and an assortment of other compounds (Shimada, T. et. al. Cancer Sci. 2004, 95, 1). Metabolism (biotransformation) of these compounds (i.e. pro-carcinogens) by CYP1 enzymes leads to the formation of carcinogenic substances. Induction of CYP1 enzymes therefore results in the biotransformation (metabolism) of PAHs to carcinogenic substances that can eventually lead to cancer. Amongst the three CYP1 enzymes, CYP1A1 has been suggested to have a role in many cancers and appears to have a major role in the genesis of lung cancer. Polymorphisms in the CYP1A2 and CYP1B1 genes have also been implicated in the risk of occurrence of certain cancers (Hu, J. Mol. Genet. Genomics. 2014, 289, 271; Xue, H. Tumour Biol. 2014, 35, 4741; Li, C. Toxicology 2015, 327, 77).

Cigarette smoke, which contains pro-carcinogenic compounds like polyaromatic hydrocarbons (PAHs) and aromatic amines, is particularly associated with the induction of CYP1A1 gene. The resultant metabolism of the PAHs in cigarette smoke is thought to be one of the primary causes of lung cancer. Recent animal and human data suggest that AhR is involved in various signaling pathways critical to cells' normal homeostasis, which includes physiological processes such as cell proliferation and differentiation, gene regulation, cell motility and migration, inflammation and others (Puga. A. et al. Biochem. Pharmacol. 2009, 77, 713). Malfunction of these processes is known to contribute to events such as tumor initiation, promotion, and progression. Therefore, using inhibitors of CYP1A1, that regulate AhR activity, for cancer chemoprevention has been considered as a promising anticancer strategy.

Like CYP1A1, the CYP1A2 enzyme is a key enzyme involved in the etiology of breast cancer by activation of carcinogenic arylamines (Ayari, I. et al. Mol. Med. Rep. 2013, 7, 280-286; Seow, A. et al. Carcinogenesis, 2001, 22, 673-677). The CYP isoform CYP1B1 is a heme-thiolate monooxygenase involved in phase I hydroxylation of many substrates including estrogens, steroids, and fatty acids which has been found to be expressed in microenvironment of almost all hormonal cancers including the prostate, ovary, mammary, uterus and pituitary, regardless of oncogenic origin, whereas it is absent in healthy tissues (Muskhelishvili, L. et al. J. Histochem. Cytochem. 2001, 49, 229-236). It is understood that CYP1B1 may have a dominant role in the genesis of hormonal mediated breast and prostate cancer (Gajjar, K. et al. Cancer Lett. 2012, 324, 13-30).

CYP1B1 inhibitors are also useful to overcome the chemo-resistance of chemotherapeutic agents. Mcfadyen and co-workers observed resistance to taxanes due to over-expression of CYP1B1, which is reversed in presence of CYP1B1 inhibitor (McFadyen, M. et al., Biochem. Pharmacol. 2001, 62, 207-212). Recently, Li and coworkers have reported CYP1B1 inhibitors and their ability to overcome docetaxel-resistance in MCF-7 cells (Cui J. et al. J. Med. Chem. 2015, 58, 3534-3547).

Reference may be made to Olguin-Reyes S. et al. Food. Chem. Toxicol. 2012, 50, 3094, Schwarz, D. et al. Eur. J. Cancer 2005, 41, 151; Urzal, R. et al. PLOS one, 2013, 8, e74917; Baumgart A. et al. Biochem. Pharmacol. 2005, 69, 657 wherein natural products khellin, bergamottin, quercetin and angellicin are reported to inhibit CYP1A1 enzyme.

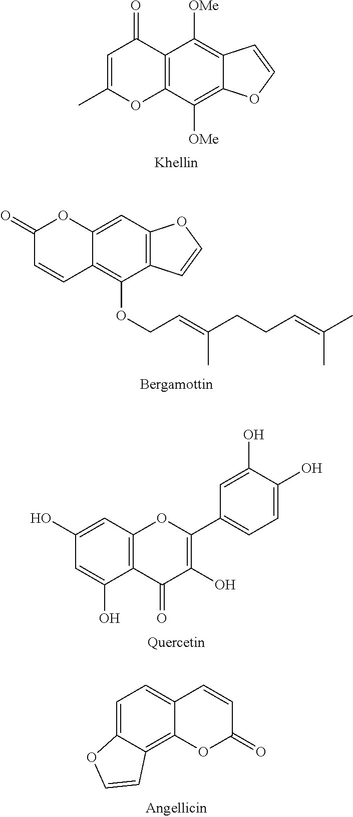 [SMILES: COc1c2occc2c(OC)c2c(=O)cc(C)oc12 ; CC(C)=CCC/C(C)=C/COc1c2ccoc2cc2oc(=O)ccc12 ; O=c1c(O)c(-c2ccc(O)c(O)c2)oc2cc(O)cc(O)c12 ; O=c1ccc2ccc3occc3c2o1]

### Objectives of the Invention

The main objective of the invention is to provide furanochalcone compounds for CYP1A1/CYP1A2/CYP1B1 inhibition activity.

Still another objective of the present invention is to provide furanochalcones for cancer chemoprevention.

Further object of the invention is to provide a process for preparation of furanochalcone compounds.

### SUMMARY OF THE INVENTION

Accordingly, the present invention relates to a compound of Formula A,

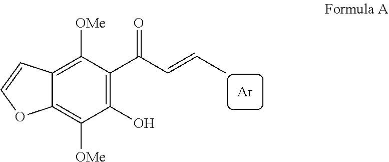

wherein, Ar is selected from the group comprising, 4-bromophenyl, 4-fluoro-3-bromo-phenyl, 2,4-difluorophenyl, 2,6-dichlorophenyl, 2-ethoxy-5-bromophenyl, 2,3-dimethoxyphenyl, 3-bromo-4-methoxyphenyl, 2,4,5-trimethoxyphenyl, thiophen-3-yl, 2,4-dichlorophenyl and anthracen-2-yl.

In an embodiment of the present invention, wherein the representative compounds comprising the following structures:

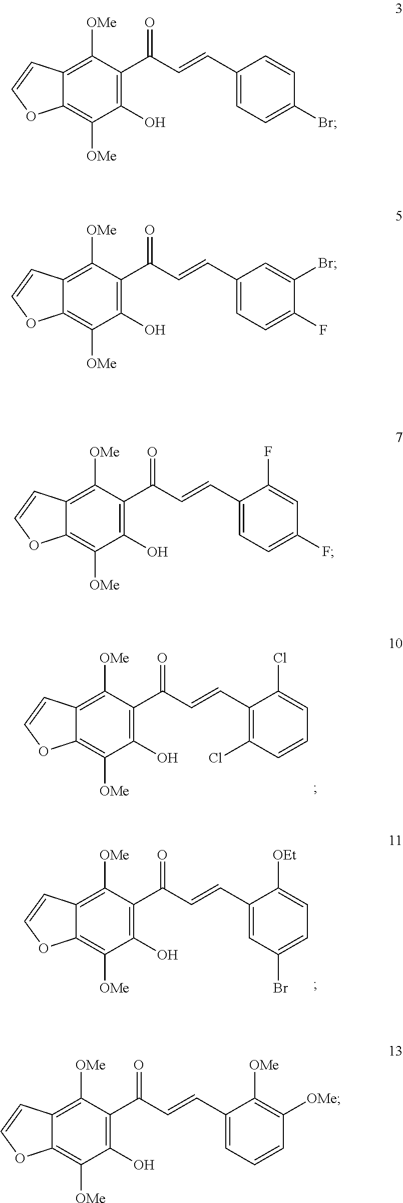 [SMILES: COc1c(C(=O)/C=C/c2ccc(Br)cc2)c(O)c(OC)c2occc12 ; COc1c(C(=O)/C=C/c2ccc(F)c(Br)c2)c(O)c(OC)c2occc12 ; COc1c(C(=O)/C=C/c2ccc(F)cc2F)c(O)c(OC)c2occc12 ; COc1c(C(=O)/C=C/c2c(Cl)cccc2Cl)c(O)c(OC)c2occc12 ; CCOc1ccc(Br)cc1/C=C/C(=O)c1c(O)c(OC)c2occc2c1OC ; COc1cccc(/C=C/C(=O)c2c(O)c(OC)c3occc3c2OC)c1OC]

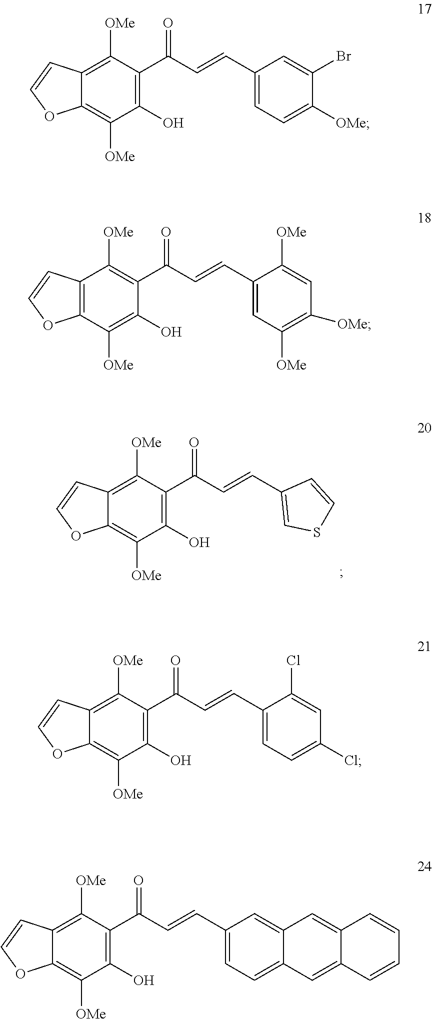 [SMILES: COc1ccc(/C=C/C(=O)c2c(O)c(OC)c3occc3c2OC)cc1Br ; COc1cc(OC)c(OC)cc1/C=C/C(=O)c1c(O)c(OC)c2occc2c1OC ; COc1c(C(=O)/C=C/c2ccsc2)c(O)c(OC)c2occc12 ; COc1c(C(=O)/C=C/c2ccc(Cl)cc2Cl)c(O)c(OC)c2occc12 ; COc1c(C(=O)/C=C/c2ccc3cc4ccccc4cc3c2)c(O)c(OC)c2occc12]

In an embodiment of the present invention, wherein the compounds are useful for the prevention or treatment of cancer caused by polyaromatic hydrocarbons (PAHs), 4-nitroquinoline-1-oxide, and N-nitroso-N-methylurea, heterocyclic amines, estrogen and 17β-estradiol; wherein the PAH is selected from a group consisting of benzo[a]pyrene (BaP), 7,12-dimethylbenz(a)anthracene (DMBA) and 2,3,7,8-tetrachlorodibenzo-p-dioxin (TCDD); heterocyclic amine is pyridine.

In another embodiment of the present invention, wherein use of the compound of formula A for prevention or treatment of cancer

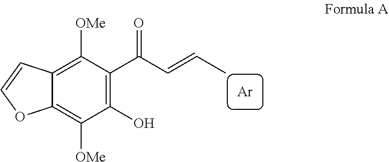

wherein, Ar is selected from the group comprising 4-bromophenyl, 4-fluoro-3-bromo-phenyl, 2,4-difluorophenyl, 2,6-dichlorophenyl, 2-ethoxy-5-bromophenyl, 2,3-dimethoxyphenyl, 3-bromo-4-methoxyphenyl, 2,4,5-trimethoxyphenyl, thiophen-3-yl, 2,4-dichlorophenyl, anthracen-2-yl, 4-chlorophenyl, 4-fluorophenyl, pyridine-3-yl, 4-methoxyphenyl, 2-chlorophenyl, 2,4-dimethoxyphenyl, pentafluorophenyl, phenyl, 3,4-methylene-dioxy-phenyl, naphth-2-yl, 2-fluorophenyl.

In yet another embodiment of the present invention, wherein use of representative compounds having Formula A

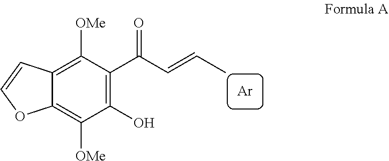

wherein, Ar is selected from the group comprising 4-bromophenyl, 4-fluoro-3-bromo-phenyl, 2,4-difluorophenyl, 2,6-dichlorophenyl, 2-ethoxy-5-bromophenyl, 2,3-dimethoxyphenyl, 3-bromo-4-methoxyphenyl, 2,4,5-trimethoxyphenyl, thiophen-3-yl, 2,4-dichlorophenyl, anthracen-2-yl, 4-chlorophenyl, 4-fluorophenyl, pyridine-3-yl, 4-methoxyphenyl, 2-chlorophenyl, 2,4-dimethoxyphenyl, pentafluorophenyl, phenyl, 3,4-methylene-dioxy-phenyl, naphth-2-yl, 2-fluorophenyl
comprising;

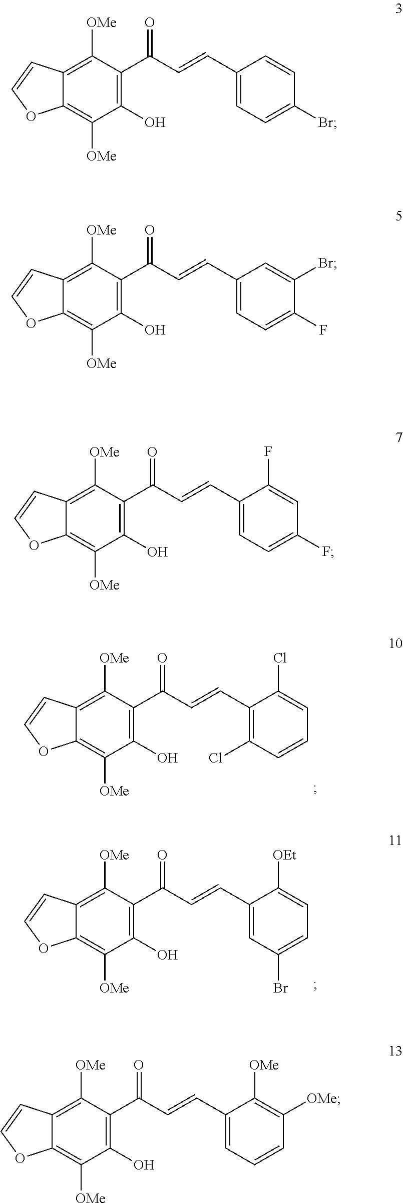 [SMILES: COc1c(C(=O)/C=C/c2ccc(Br)cc2)c(O)c(OC)c2occc12 ; COc1c(C(=O)/C=C/c2ccc(F)c(Br)c2)c(O)c(OC)c2occc12 ; COc1c(C(=O)/C=C/c2ccc(F)cc2F)c(O)c(OC)c2occc12 ; COc1c(C(=O)/C=C/c2c(Cl)cccc2Cl)c(O)c(OC)c2occc12 ; CCOc1ccc(Br)cc1/C=C/C(=O)c1c(O)c(OC)c2occc2c1OC ; COc1cccc(/C=C/C(=O)c2c(O)c(OC)c3occc3c2OC)c1OC]

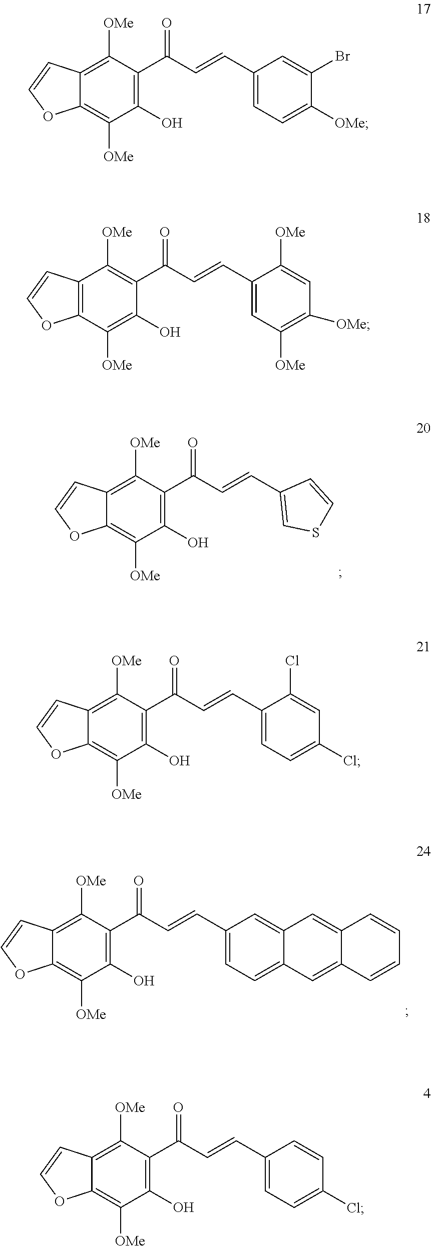 [SMILES: COc1ccc(/C=C/C(=O)c2c(O)c(OC)c3occc3c2OC)cc1Br ; COc1cc(OC)c(OC)cc1/C=C/C(=O)c1c(O)c(OC)c2occc2c1OC ; COc1c(C(=O)/C=C/c2ccsc2)c(O)c(OC)c2occc12 ; COc1c(C(=O)/C=C/c2ccc(Cl)cc2Cl)c(O)c(OC)c2occc12 ; COc1c(C(=O)/C=C/c2ccc3cc4ccccc4cc3c2)c(O)c(OC)c2occc12 ; COc1c(C(=O)/C=C/c2ccc(Cl)cc2)c(O)c(OC)c2occc12]

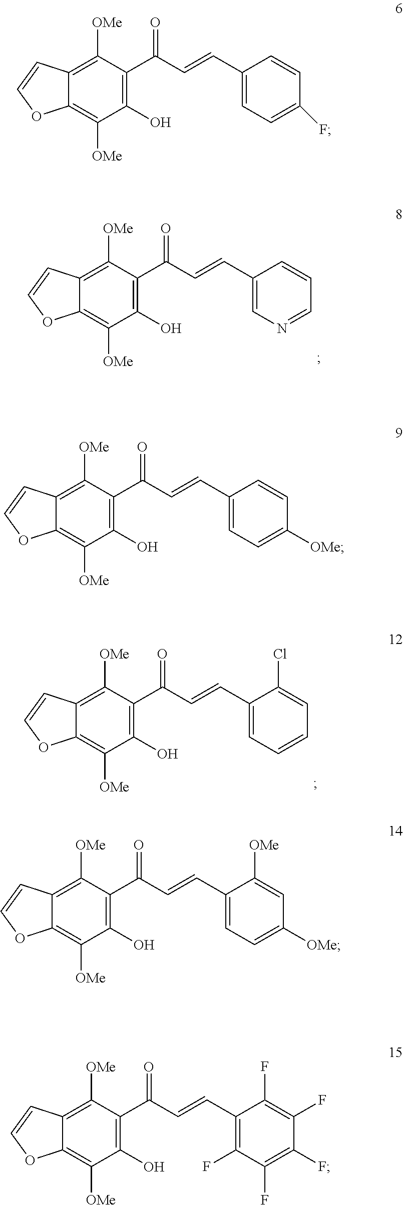 [SMILES: COc1c(C(=O)/C=C/c2ccc(F)cc2)c(O)c(OC)c2occc12 ; COc1c(C(=O)/C=C/c2cccnc2)c(O)c(OC)c2occc12 ; COc1ccc(/C=C/C(=O)c2c(O)c(OC)c3occc3c2OC)cc1 ; COc1c(C(=O)/C=C/c2ccccc2Cl)c(O)c(OC)c2occc12 ; COc1ccc(/C=C/C(=O)c2c(O)c(OC)c3occc3c2OC)c(OC)c1 ; COc1c(C(=O)/C=C/c2c(F)c(F)c(F)c(F)c2F)c(O)c(OC)c2occc12]

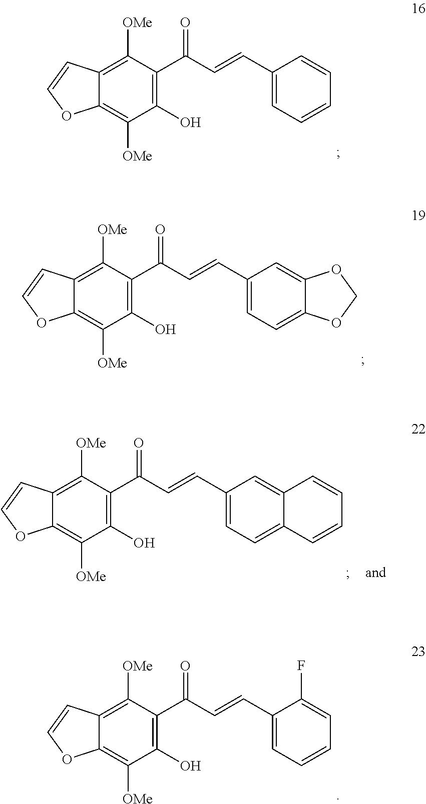 [SMILES: COc1c(C(=O)/C=C/c2ccccc2)c(O)c(OC)c2occc12 ; COc1c(C(=O)/C=C/c2ccc3c(c2)OCO3)c(O)c(OC)c2occc12 ; COc1c(C(=O)/C=C/c2ccc3ccccc3c2)c(O)c(OC)c2occc12 ; COc1c(C(=O)/C=C/c2ccccc2F)c(O)c(OC)c2occc12]

In still another embodiment of the present invention, wherein the use of compound to overcoming the chemo-resistance to cisplatin, docetaxel and paclitaxel through inhibition of CYP1B1.

In a preferred embodiment of the present invention, wherein IC₅₀ value of compound 8 is 342 and 470 nM against CYP1A1 in Saccharosomes and in live cells.

In another preferred embodiment of the present invention, wherein a process for preparation of compound of Formula A wherein the process comprising the steps of:
a) reacting khellin with alkali hydroxide in an alcohol at reflux temperature ranging between 80-120° C. over a period in the range of 12-14 hours followed by concentrating the reaction mixture and extracting with an aqueous solvent. selected from a group consisting of DCM: H₂O, chloroform: H₂O, or acetone: H₂O to obtain khellinone (2);
b) reacting khellinone (2) obtained in step (a) with an aldehydes in presence of catalytic amount of alkali selected from KOH or NaOH in alcohol selected from a group consisting of methanol or ethanol at a temperature in the range of 0° C. to 1° C. over a period ranging between 12-14 hours to obtain compound of Formula A as claimed in claim 1.

In another preferred embodiment of the present invention, wherein alkali hydroxide used in step (a) is selected from a group consisting of Sodium hydroxide and Potassium hydroxide.

In a preferred embodiment of the present invention, wherein the alcohol used in step (a) is selected from a group consisting of ethanol and methanol.

In another preferred embodiment of the present invention, wherein the aldehyde used in step (b) is selected from a group consisting of 4-bromophenyl aldehyde, 4-fluoro-3-bromo-phenyl aldehyde, 2,4-difluorophenyl aldehyde, 2,6-dichlorophenyl aldehyde, 2-ethoxy-5-bromophenyl aldehyde, 2,3-dimethoxyphenyl aldehyde, 3-bromo-4-methoxyphenyl aldehyde, 2,4,5-trimethoxyphenyl aldehyde, thiophen-3-yl aldehyde, 2,4-dichlorophenyl aldehyde and anthracen-2-yl aldehyde, 4-chlorophenyl aldehyde, 4-fluorophenyl aldehyde, pyridine-3-yl aldehyde, 4-methoxyphenyl aldehyde, 2-chlorophenyl aldehyde, 2,4-dimethoxyphenyl aldehyde, pentafluorophenyl aldehyde, phenyl aldehyde, 3,4-methylene-dioxy-phenyl aldehyde, naphth-2-yl aldehyde, 2-fluorophenyl aldehyde.

In yet another embodiment of the present invention, wherein a pharmaceutical composition for the prevention or treatment of cancer comprising an effective amount of the compound of structural Formulae A as claimed in claim 1 individually or in combination thereof, optionally, along with the pharmaceutically acceptable excipients, diluents.

In still another embodiment of the present invention, wherein the pharmaceutically acceptable excipient are saccharides selected from lactose, starch, dextrose, stearates selected from stearic acid, magnesium stearate, polyvinyl pyrrolidine, dicalcium phosphate dihydrate, eudragit polymers, celluloses, polyethylene glycol, polysorbate 80, sodium lauryl sulfate, magnesium oxide, silicon dioxide, carbonates selected from sodium carbonate, sodium bicarbonate and talc.

In another embodiment of the invention, the representative compounds comprising the structural formulae:

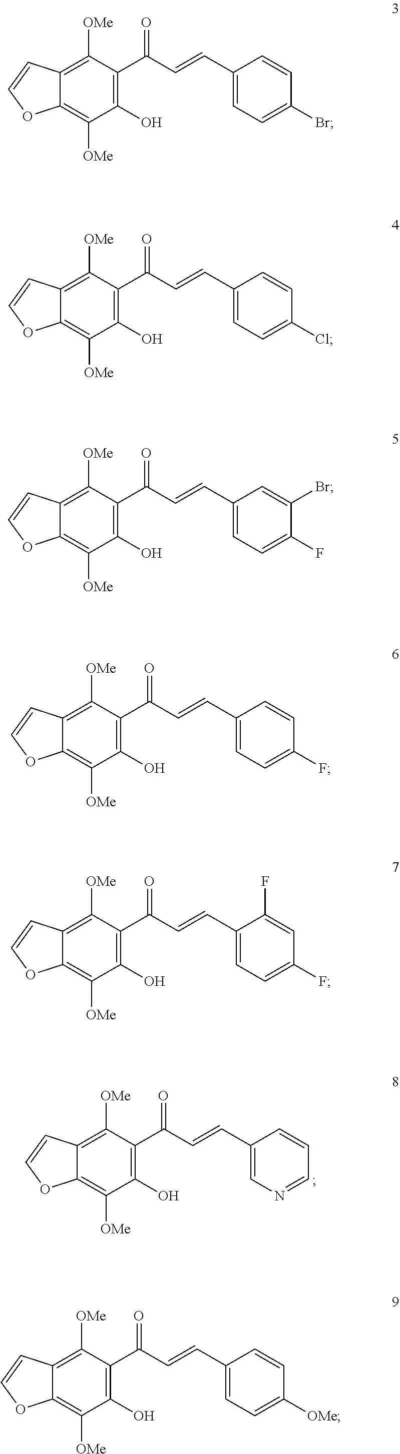

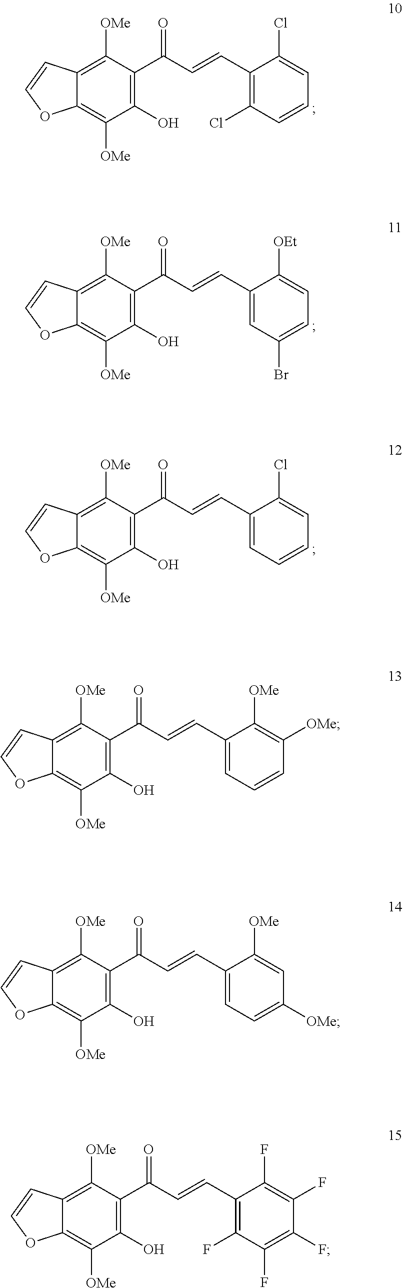 [SMILES: COc1c(C(=O)/C=C/c2c(Cl)cccc2Cl)c(O)c(OC)c2occc12 ; CCOc1ccc(Br)cc1/C=C/C(=O)c1c(O)c(OC)c2occc2c1OC ; COc1c(C(=O)/C=C/c2ccccc2Cl)c(O)c(OC)c2occc12 ; COc1cccc(/C=C/C(=O)c2c(O)c(OC)c3occc3c2OC)c1OC ; COc1ccc(/C=C/C(=O)c2c(O)c(OC)c3occc3c2OC)c(OC)c1 ; COc1c(C(=O)/C=C/c2c(F)c(F)c(F)c(F)c2F)c(O)c(OC)c2occc12]

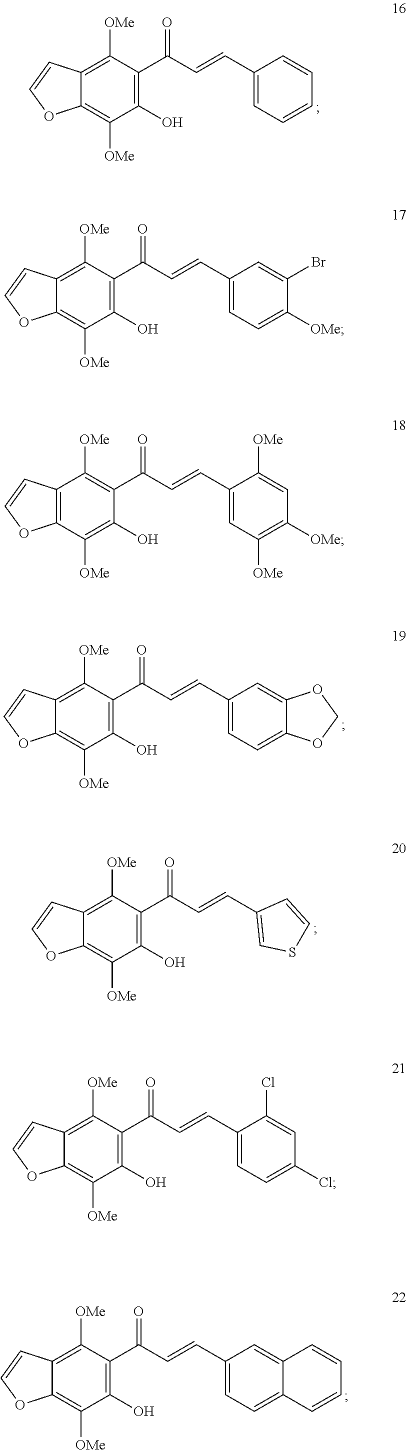

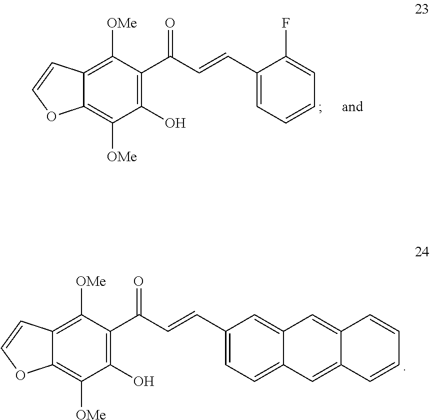 [SMILES: COc1c(C(=O)/C=C/c2ccccc2F)c(O)c(OC)c2occc12 ; COc1c(C(=O)/C=C/c2ccc3cc4ccccc4cc3c2)c(O)c(OC)c2occc12]

In another embodiment of the invention, a method is presented for preventing carcinogenesis in a patient suffering or at a risk of developing carcinogenesis by administering the composition of above mentioned compounds of formula I at therapeutically-effective dose.

In another embodiment of the invention, above described compounds are useful for the prevention of cancer caused by the carcinogens such as polyaromatic hydrocarbons (PAHs), 4-nitroquinoline-1-oxide, and N-nitroso-N-methylurea, heterocyclic amines, estrogen and 17β-estradiol. Examples of PAH are benzo[a]pyrene (BaP), 7,12-dimethylbenz(a)anthracene (DMBA) and 2,3,7,8-tetrachlorodibenzo-p-dioxin (TCDD). Example of heterocyclic amine is pyridine.

In one more embodiment of the invention, most active representative compounds 8 display IC₅₀ of 342 and 470 nM for CYP1A1 inhibition in Saccharosomes and in HEK293 cells (transfected with the pcDNA3.1/CYP1A1), are useful for the prevention of cancers which could be caused by imbibing polyaromatic hydrocarbons such as the known carcinogens B[a]P, TCDD or DMBA.

In another embodiment of the invention, a process is described for the preparation of the khellinone derivatives 3-24, wherein
i. Khellinone (2) is prepared from khellin (a furochromone) by reacting khellin and potassium hydroxide (or sodium hydroxide) in round-bottom flask in ethanol (or methanol) at reflux temperature of 80-120° C. ° C. over a period of 12-14 h. Furthermore, the reaction mixture is concentrated and extracted with DCM: H₂O, chloroform: H₂O, or acetone: H₂O. Organic layer is collected and concentrated on rotary evaporator to get crude product, which on silica gel column chromatography (5-10% ethyl acetate in hexane) gave khellinone (2) as a yellow powder.
ii. Khellinone (2) was then reacted with different aldehydes in presence of catalytic amount of 1 M of KOH (or NaOH) in 50 ml ethanol (or methanol) at a temperature of 0° C. to 1° C. over a period of 12-14 h. Reaction mixture was concentrated in vacuum and residue is extracted with DCM: H₂O. Organic layer is separated on silica gel column chromatography (5-25%) and concentrated on rotary evaporator to get crude product to obtain products 3-24.

In another embodiment of the invention, a pharmaceutical composition for the prevention of cancer and related diseases comprising an effective amount of the compound of general formula I, optionally, along with the pharmaceutically acceptable excipients or diluents which are useful for the prevention of cancers caused by imbibing polyaromatic hydrocarbons, such as the known carcinogens BaP, TCDD or DMBA.

In another embodiment of the invention, wherein the pharmaceutically acceptable excipient is selected from a group consisting of saccharides (such as lactose, starch, dextrose), stearates (such as stearic acid, magnesium stearate), polyvinyl pyrrolidine, dicalcium phosphate dihydrate, eudragit polymers, celluloses, polyethylene glycol, polysorbate 80, sodium lauryl sulfate, magnesium oxide, silicon dioxide, carbonates (such as sodium carbonate, sodium bicarbonate), talc are useful for the prevention or treatment of cancers caused by imbibing polyaromatic hydrocarbons, such as known carcinogens BaP, TCDD or DMBA.

### BRIEF DESCRIPTION OF ACCOMPANYING DRAWINGS

FIG. 1 is a diagram illustrating the chemical synthesis of furanochalcones 3-24 claimed in the invention.

FIG. 2 shows molecular modeling images depicting interactions of most potent compounds with CYP1A1 and CYP1A2. (A). Interactions of α-naphthoflavone with CYP1A1; (B) Interactions of compound 8 with CYP1A1; (C) Interactions of α-naphthoflavone with CYP1A2; (D) Interactions of compound 8 with CYP1A2.

FIG. 3 shows the dose-response curves of compound 8 for inhibition of CYP1A1, CYP1A2, CYP1B1, CYP3A4 and CYP2D6 in Saccharosomes (yeast microsomes).

FIG. 4 shows the dose-response curves of compounds 6 and 8 for inhibition of CYP1A1 and CYP1B1 in human live cells (HEK293 cells)

### LIST OF ABBREVIATIONS

PAHs: polyaromatic hydrocarbons; CYP1A1: Cytochrome P4501A1; CYP1B1: Cytochrome P4501B1; BaP: Benzo[a]pyrene; TCDD: 2,3,7,8-tetrachlorodibenzo-p-dioxin; DMBA: 7,12-Dimethylbenz(a)anthracene.

### DETAILED DESCRIPTION OF THE INVENTION

The present invention reports furanochalcone class of compounds represented by the general formula A as promising CYP1A1, CYP1A2 and CYP1B1 inhibitors.

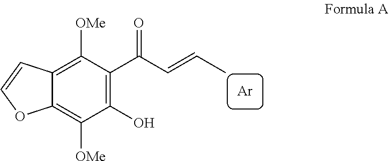

The present invention relates to furanochalcones that showed promising CYP1A1 inhibitory activity in both in-vitro microsomes and live cells. The results of compounds 3-24 for CYP1A1 inhibition activity in Saccharosomes™ are depicted in Table 1. Furthermore, the CYP1A1 and CYP1A2 inhibitory potential of all compounds was tested in live cell assay of CYP1A1 enzyme in HEK293 cells transfected with the pcDNA3.1/CYP1A1 against 5 μM EROD and CYP1A2 in HEK293 cells transfected with the pcDNA3.1/CYP1A2 against 5 μM EROD. Most promising compound 8 displayed IC₅₀ of 342 and 470 nM against CYP1A1 in Saccharosomes and in live cells (Table 3 and 5).

Compounds of the invention derived from formula but are not limited to the following chemical structures:

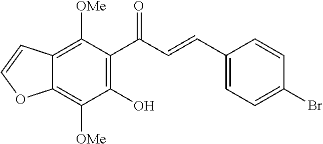 [SMILES: COc1c(C(=O)/C=C/c2ccc(Br)cc2)c(O)c(OC)c2occc12]

### 3-(4,7-dimethoxy-6-hydroxybenzofuran-5-yl)-1-(4-bromophenyl)-3-oxopropene (3)

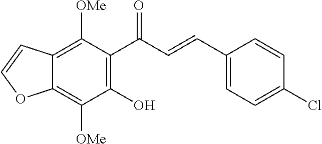 [SMILES: COc1c(C(=O)/C=C/c2ccc(Cl)cc2)c(O)c(OC)c2occc12]

### 3-(4,7-dimethoxy-6-hydroxybenzofuran-5-yl)-1-(4-chlorophenyl)-3-oxopropene (4)

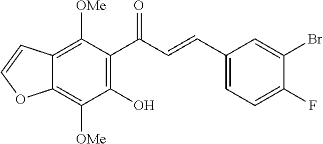 [SMILES: COc1c(C(=O)/C=C/c2ccc(F)c(Br)c2)c(O)c(OC)c2occc12]

### 3-(4,7-dimethoxy-6-hydroxybenzofuran-5-yl)-1-(3-bromo-4-flurophenyl)-3-oxopropene (5)

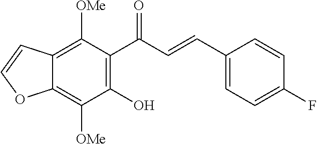 [SMILES: COc1c(C(=O)/C=C/c2ccc(F)cc2)c(O)c(OC)c2occc12]

### 3-(4,7-dimethoxy-6-hydroxybenzofuran-5-yl)-1-(4-fluorophenyl)-3-oxopropene (6)

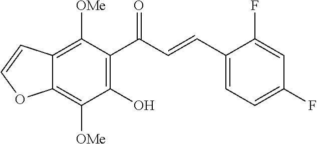 [SMILES: COc1c(C(=O)/C=C/c2ccc(F)cc2F)c(O)c(OC)c2occc12]

### 3-(4,7-dimethoxy-6-hydroxybenzofuran-5-yl)-1-(2,4-difluoroflurophenyl)-3-oxopropene (7)

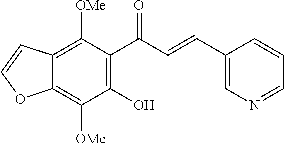 [SMILES: COc1c(C(=O)/C=C/c2cccnc2)c(O)c(OC)c2occc12]

### 3-(4,7-dimethoxy-6-hydroxybenzofuran-5-yl)-1-(pyridin-3-yl)-3-oxopropene (8)

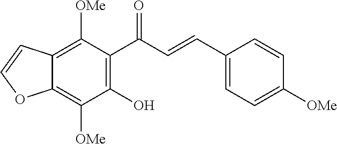 [SMILES: COc1ccc(/C=C/C(=O)c2c(O)c(OC)c3occc3c2OC)cc1]

### 3-(4,7-dimethoxy-6-hydroxybenzofuran-5-yl)-1-(4-methoxyphenyl)-3-oxopropene (9)

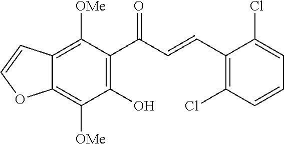 [SMILES: COc1c(C(=O)/C=C/c2c(Cl)cccc2Cl)c(O)c(OC)c2occc12]

### 3-(4,7-dimethoxy-6-hydroxybenzofuran-5-yl)-1-(2,6-dichlorophenyl)-3-oxopropene (10)

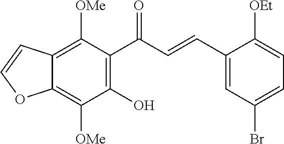 [SMILES: CCOc1ccc(Br)cc1/C=C/C(=O)c1c(O)c(OC)c2occc2c1OC]

### 3-(4,7-dimethoxy-6-hydroxybenzofuran-5-yl)-1-(2-ethoxy,5-bromophenyl)-3-oxopropene (11)

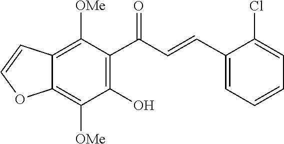 [SMILES: COc1c(C(=O)/C=C/c2ccccc2Cl)c(O)c(OC)c2occc12]

### 3-(4,7-dimethoxy-6-hydroxybenzofuran-5-yl)-1-(2-ethoxy,5-bromophenyl)-3-oxopropene (12)

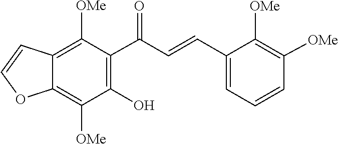 [SMILES: COc1cccc(/C=C/C(=O)c2c(O)c(OC)c3occc3c2OC)c1OC]

### 3-(4,7-dimethoxy-6-hydroxybenzofuran-5-yl)-1-(2,3 dimethoxyphenyl)-3-oxopropene (13)

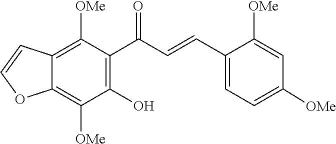 [SMILES: COc1ccc(/C=C/C(=O)c2c(O)c(OC)c3occc3c2OC)c(OC)c1]

### 3-(4,7-dimethoxy-6-hydroxybenzofuran-5-yl)-1-(2,3 dimethoxyphenyl)-3-oxopropene (14)

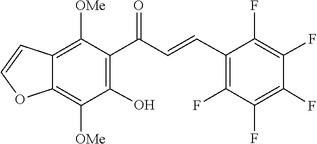 [SMILES: COc1c(C(=O)/C=C/c2c(F)c(F)c(F)c(F)c2F)c(O)c(OC)c2occc12]

### 3-(4,7-dimethoxy-6-hydroxybenzofuran-5-yl)-1-(2,3,4,5,6 pentafluorophenyl)-3-oxopropene (15)

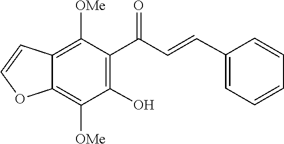 [SMILES: COc1c(C(=O)/C=C/c2ccccc2)c(O)c(OC)c2occc12]

### 3-(4,7-dimethoxy-6-hydroxybenzofuran-5-yl)-1-(phenyl)-3-oxopropene (16)

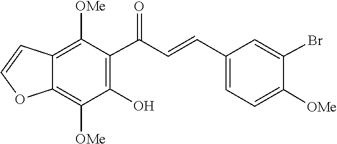 [SMILES: COc1ccc(/C=C/C(=O)c2c(O)c(OC)c3occc3c2OC)cc1Br]

### 3-(4,7-dimethoxy-6-hydroxybenzofuran-5-yl)-1-(3-bromo-4-methoxyphenyl)-3-oxopropene (17)

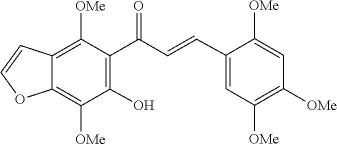 [SMILES: COc1cc(OC)c(OC)cc1/C=C/C(=O)c1c(O)c(OC)c2occc2c1OC]

### 3-(4,7-dimethoxy-6-hydroxybenzofuran-5-yl)-1-(2,4,5-trithoxyphenyl)-3-oxopropene (18)

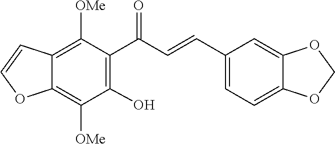 [SMILES: COc1c(C(=O)/C=C/c2ccc3c(c2)OCO3)c(O)c(OC)c2occc12]

### 3-(benzo[d][1,3]dioxol-5-yl)-1-(6-hydroxy-4,7-dimethoxybenzofuran-5-yl)prop-2-en-1-one (19)

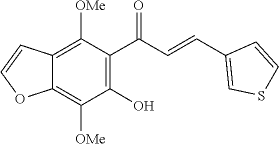 [SMILES: COc1c(C(=O)/C=C/c2ccsc2)c(O)c(OC)c2occc12]

### 1-(6-hydroxy-4,7-dimethoxybenzofuran-5-yl)-3-(thiophen-3-yl)prop-2-en-1-one (20)

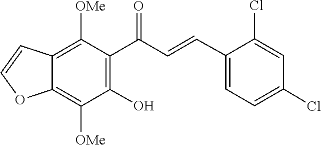 [SMILES: COc1c(C(=O)/C=C/c2ccc(Cl)cc2Cl)c(O)c(OC)c2occc12]

### 3-(2,4-dichlorophenyl)-1-(6-hydroxy-4,7-dimethoxybenzofuran-5-yl)prop-2-en-1-one (21)

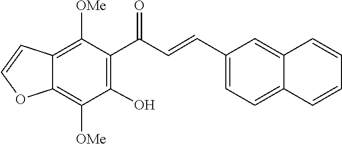 [SMILES: COc1c(C(=O)/C=C/c2ccc3ccccc3c2)c(O)c(OC)c2occc12]

### 1-(6-hydroxy-4,7-dimethoxybenzofuran-5-yl)-3-(naphthalen-2-yl)prop-2-en-1-one (22)

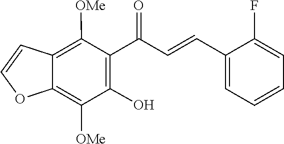 [SMILES: COc1c(C(=O)/C=C/c2ccccc2F)c(O)c(OC)c2occc12]

### 3-(2-fluorophenyl)-1-(6-hydroxy-4,7-dimethoxybenzofuran-5-yl)prop-2-en-1-one (23)

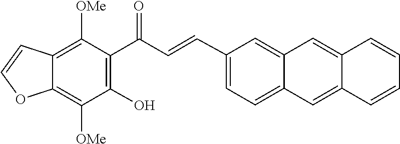 [SMILES: COc1c(C(=O)/C=C/c2ccc3cc4ccccc4cc3c2)c(O)c(OC)c2occc12]

### 3-(anthracen-2-yl)-1-(6-hydroxy-4,7-dimethoxybenzofuran-5-yl)prop-2-en-1-one (24)

As used herein, the terms below have the meanings indicated.

The term aryl as used herein, alone or in combination, means a carbocyclic aromatic system containing one, two or three rings wherein such rings may be attached together in a pendant manner or may be fused, optionally, substituted with at least one halogen, an alkyl containing from 1 to 3 carbon atoms, an alkoxyl, an aryl radical, a nitro function, a polyether radical, a heteroaryl radical, a benzoyl radical, an alkyl ester group, a carboxylic acid, a hydroxyl optionally protected with an acetyl or benzoyl group, or an amino function optionally protected with an acetyl or benzoyl group or optionally substituted with at least one alkyl containing from 1 to 12 carbon atoms.

The compounds of the invention can be used to treat a patient (e.g. a human) that suffers from or is at a risk of suffering from a disease, disorder, condition, or symptom described herein. The compounds of the invention can be used alone or in combination with other agents and compounds in methods of treating or preventing cancer or related diseases. Each such treatment described above includes the step of administering to a patient in need thereof a therapeutically effective amount of the compound of the invention described herein to delay, reduce or prevent such a disease, disorder, condition, or symptom.

It is understood that the foregoing examples are merely illustrative of the present invention. Certain modifications of the articles and/or methods employed may be made and still achieve the objectives of the invention. Such modifications are contemplated as within the scope of the claimed invention.

### Examples

The following examples are given by way of illustration of the working of the invention in actual practice and should not be construed to limit the scope of the present invention in any way.

### Example 1: Synthesis of 3-(4,7-dimethoxy-6-hydroxybenzofuran-5-yl)-1-(4-bromophenyl)-3-oxopropene (3). Scheme is shown in FIG. 1

Step 1: Synthesis of 1-(6-hydroxy-4,7-dimethoxybenzofuran-5-yl)ethanone (2, khellinone). Khellin (1) was purchased from Sigma (product number 286419; CAS number: 82-02-0). Khellin (900 mg) was treated with the catalytic amount of 1 M potassium hydroxide in 10 ml ethanol at reflux temperature of 90° C. over a period of 12-14 hr. The reaction mixture was concentrated and residue was extracted with DCM: H₂O. Organic layer was collected and concentrated on rotary evaporator to get crude product, which on silica gel column chromatography (5-10% ethyl acetate in hexane) gave 1-(6-hydroxy-4,7-dimethoxybenzofuran-5-yl)ethanone (2, 590 mg) as a yellow powder. yellow crystals; HPLC: t_{R}=4.6 min (99% purity); yield: 95%; m.p. 169-170° C.; IR (CHCl₃): νₘₐₓ 3436, 3160, 3137, 2989, 2931, 2960, 2830, 1619, 1586, 1471, 1444, 1424, 1364, 1380, 1300, 1265 cm⁻¹ H NMR (400 MHz, CDCl₃): δ (ppm) 7.51 (d, 1H, J=2.2 Hz, CH), 6.91 (d, 1H, J=4.0 Hz, CH), 4.15 (s, 3H, OMe), 4.05 (s, 3H, OMe), 2.73 (s, 3H, Me); ¹³C NMR (100 MHz, CDCl₃): δ (ppm) 206.2 (C═O), 153.5 (C-7a), 152.3 (C-6), 151.6 (C-3), 143.8 (OCH═CH), 128.8 (C-7), 110.8 (C-3a), 110.5 (C-5), 106.7 (OCH═C̅H), 61.0 (OMe), 60.9 (OMe), 33.2 (C-Me), HR-ESIMS: m/z 237.0759 [M+H]⁺ calcd for C₁₂H₁₂O₅+H+(237.0757).

Step 2: Procedure for synthesis of 3-(4,7-dimethoxy-6-hydroxybenzofuran-5-yl)-1-(4-bromophenyl)-3-oxopropene (3): 1-(6-Hydroxy-4,7-dimethoxybenzofuran-5-yl)ethanone (2, 80 mg) obtained in step 1 was reacted with 4-bromo benzaldehyde in presence of catalytic amount of 1M of KOH in 50 ml methanol at a temperature 0° C. over a period of 12-14 hr. reaction mixture is concentrated in vacuum and residue is extracted with DCM: H₂O. Organic layer is collected and concentrated on rotary evaporator to get crude product which on silica gel column chromatography (5-25%) gave pure compound (3, 24 mg). White solid; HPLC: t_{R}=4.5 min (100% purity) yield: 88%; m.p. 135-137° C.; IR (CHCl₃): νₘₐₓ 3400, 2919, 2850, 1682, 1613, 1544, 1414, 1389, 1435, 1349, 1279, 112, 1012, 909; ¹H NMR (400 MHz, CDCl₃): ¹H NMR (400 MHz, CDCl₃): δ (ppm) 7.82 (d, 2H, J=12.0 Hz, CH), 7.58 (d, 1H, J=8.0 Hz, 1H, CH), 7.53 (d, 1H, J=4.0 Hz, OCH═CH), 7.40 (d, 2H, J=12.0 Hz, CH), 6.89 (d, 1H, J=4.0 Hz, OCH═CH), 4.09 (s, 3H, OMe), 4.04 (s, 3H, OMe); ¹³C NMR (100 MHz, CDCl₃): 194.4 (C═O), 153.1 (C-6), 152.1 (C-9a), 150.7 (C-4), 144.3 (OCH═CH), 141.4, 137.3, 133.1, 130.9, 130.5, 127.2, 123.1, 112.7, 111.7, 106.2 (OCH═CH), 62.0, 61.0; HR-ESIMS 403.0175 [M+H]⁺ calcd for C₁₉H₁₅BrO₅+H+(403.0175).

Example 2: Synthesis of 3-(4, 7-dimethoxy-6-hydroxybenzofuran-5-yl)-1-(4-chlorophenyl)-3-oxopropene (4). Procedure for synthesis of 3-(4,7-dimethoxy-6-hydroxybenzofuran-5-yl)-1-(4-chlorophenyl)-3-oxopropene (4) is similar to example number 1 (steps 1 and 2) except the respective starting material 4-chloro benzaldehyde is used in step 2. Orange crystals; HPLC: t_{R}=49.6 min (90% purity); yield: 95%; m.p. 162-164° C.; IR (CHCl₃): νₘₐₓ 3448, 3053, 2928, 2868, 2304, 1730, 1656, 1619, 1585, 1462, 1386, 1327, 1313, 12679, 1262, 1210, 1149, 1039; ¹H NMR (400 MHz, CDCl₃): δ (ppm) 7.98 (d, 2H, J=4.0 Hz, CH), 7.52 (d, 1H, J=4.0 Hz OCH═CH), 7.40 (d, J=8.0 Hz, 2H, CH), 7.19 (m, 2H, CH), 6.88 (d, 1H, J=4.0 Hz, OCH═CH), 4.09 (s, 3H, OMe), 4.05 (s, 3H, OMe); ¹³C NMR (100 MHz, CDCl₃): δ (ppm) 194.5 (C═O), 153.2 (C-6), 152.0 (C-9a), 150.7 (C-4), 144.2 (OCH═CH), 141.9, 136.3, 133.6, 129.6, 129.3, 127.5, 127.4 112.7, 111.8, 106.2 (OCH═CH), 62.0, 61.0; HR-ESIMS: m/z 359.0677[M+H]⁺ calcd for C₁₉H₁₅ ClO₅+H⁺ (359.0608).

Example 3: Synthesis of 3-(4, 7-dimethoxy-6-hydroxybenzofuran-5-yl)-1-(3-bromo-4-fluorophenyl)-3-oxopropene (5). Procedure of synthesis is similar to example number 1 (steps 1 and 2) except the respective starting material 3-bromo-4-fluorobenzaldheyde is used in step 2. white solid; HPLC: t_{R}=5.0 min (97% purity); yield: 94%; m.p. 186-188° C.; IR (CHCl₃): νₘₐₓ 3400, 2921, 2850, 1630, 1557, 1494, 1463, 1442, 1417, 1382, 1359, 1332, 1299, 1269, 1151, 1091; ¹H NMR (400 MHz, CDCl₃): δ (ppm) 7.85 (m, 1H, CH), 7.76 (d, 2H, J=8.0 Hz, CH), 7.54 (t, 2H, J=4.0 Hz, CH), 7.12 (t, 1H, J=12.0 Hz, CH), 6.89 (d, 1H, J=2.2 Hz, OCH═CH), 4.09 (s, 3H, OMe), 4.05 (s, 3H, OMe); ¹³C NMR (100 MHz, CDCl₃): δ (ppm) 194.5 (C═O), 165.3, 162.8, 153.1, 151.9, 150.6, 144.2 (OCH═CH), 142.2, 131.4, 130 0.4, 130.3, 129.6, 126.7, 116.2, 112.8, 111.9, 106.1 (OCH═CH), 62.0, 61.0; HR-ESIMS: m/z 423.0060 [M+H]⁺ calcd for C₁₉H₁₄ BrFO₅+H⁺ (423.0060).

Example 4: Synthesis of 3-(4, 7-dimethoxy-6-hydroxybenzofuran-5-yl)-1-(4-fluorophenyl)-3-oxopropene (6). Procedure of synthesis is similar to example number 1 (steps 1 and 2) except the respective starting material 4-fluorobenzaldheyde is used in step 2. Cream colored oil; HPLC: t_{R}=48.4 min (98% purity); yield: 90%; IR (CHCl₃): νₘₐₓ 3400, 2922, 2851, 1628, 1601, 1556, 1544, 1510, 1461, 1443, 1413, 1360, 1297, 1299, 1266, 1151, 1091; ¹H NMR (400 MHz, CDCl₃): δ (ppm) 7.82 (s, 2H, CH), 7.64 (dd, 2H, J=8.0 Hz, J=5.5 Hz, CH), 7.53 (d, 1H, J=2.2 Hz, OCH═CH), 7.12 (t, 2H, J=8.6 Hz, CH), 6.88 (d, 1H, J=2.3 Hz, OCH═CH), 4.09 (s, 3H, OMe), 4.05 (s, 3H, OMe); ¹³C NMR (100 MHz, CDCl₃): δ (ppm) 194.5 (C═O), 165.0, 163.0, 153.1, 151.9, 150.7, 144.2 (OCH═CH), 142.2, 131.4, 130.4, 126.7, 116.2, 112.8, 111.8, 106.2 (OCH═CH), 62.0, 61.0, HR-ESIMS: m/z 365.0838 [M+Na]⁺ calcd for C₁₉H₁₅ FNaO5 (365.0801).

Example 5: Synthesis of 3-(4,7-dimethoxy-6-hydroxybenzofuran-5-yl)-1-(2,4-difluorophenyl)-3-oxopropene (7). Procedure of synthesis is similar to example number 1 (steps 1 and 2) except the respective starting material 2,4-difluorobenzaldheyde is used in step 2. White solid; HPLC: t_{R}=42.4 min (98% purity); yield: 90%, m.p. 154-156° C.; IR (CHCl₃): νₘₐₓ 3400, 2919, 2850, 1633, 1618, 1588, 1562, 1542, 1464, 1438, 1412, 1377, 1357, 1286, 1211, 1153, 1119; ¹H NMR (400 MHz, CDCl₃): δ (ppm) 7.83 (d, 1H, J=16.0 Hz, CH), 7.72 (d, 1H, J=12.0 Hz, CH), 7.54 (d, 1H, J=2.2 Hz, OCH═CH), 7.14 (d, 2H, J=4.0 Hz, CH), 6.89 (d, 1H, J=4.0 Hz, CH), 6.86 (d, 1H, J=4.0 Hz, OCH═CH), 4.09 (s, 3H, OMe), 4.07 (s, 3H, OMe); ¹³C NMR (100 MHz, CDCl₃): δ (ppm) 194.2 (C═O), 164.8, 162.0, 153.2, 152.2, 150.8, 144.3 (OCH═CH), 140.2, 138.5, 129.5, 112.5, 111.6, 111.0, 110.9, 110.7, 105.4, 105.2 (OCH═CH), 61.9, 61.0; HR-ESIMS: m/z 361.0912 [M+H]⁺ calcd for C₁₉H₁₄F₂O₅+H⁺ (361.0882).

Example 6: Synthesis of 3-(4, 7-dimethoxy-6-hydroxybenzofuran-5-yl)-1-(pyridine-2-yl)-3-oxopropene (8). Procedure of synthesis is similar to example number 1 (steps 1 and 2) except the respective starting material pyridine-2-carbaxaldehyde is used in step 2. Orange crystals; HPLC: t_{R}=49.6 min (95%); yield: 85%; m.p. 112-114° C.; IR (CHCl₃): 3400, 2919, 2850, 1633, 1618, 1588, 1562, 1542, 1464, 1438, 1412, 1377, 1357, 1286, 1211, 1153, 1119; ¹H NMR (400 MHz, CDCl₃): δ (ppm) 8.88 (s, 1H, CH), 8.63 (d, 1H, J=4.0 Hz, CH), 7.94 (m, 2H, CH), 7.81 (d, J=16.0 Hz, CH), 7.54 (d, 1H, J=2.3 Hz, OCH═CH), 7.37 (dd, 1H, J=8.0 Hz, J=4.9 Hz, CH), 6.89 (d, 1H, J=2.3 Hz, OCH═CH), 4.09 (s, 3H, OMe), 4.06 (s, 3H, OMe); ¹³C NMR (100 MHz, CDCl₃): δ (ppm) 194.2 (C═O), 153.2, 152.2, 150.9, 149.8, 144.3, 139.3, 134.8, 131.0, 128.9, 123.8, 112.5, 112.5, 111.6, 111.5, 106.3, 61.8, 61.1; HR-ESIMS: m/z 326.1033 [M+H]⁺ calcd for C₁₈H₁₅NO₅+H⁺ (326.1023).

Example 7: Synthesis of 3-(4, 7-dimethoxy-6-hydroxybenzofuran-5-yl)-1-(4-methoxyphenyl)-3-oxopropene (9). Procedure of synthesis is similar to example number 1 (step land 2) except the respective starting material 4-methoxybenzaldehyde is used in step 2. yellow crystals; HPLC: t_{R}=5.3 min (100% purity); yield: 92%; m.p. 198-199° C. IR (CHCl₃): νₘₐₓ 3435, 2923, 2851, 1630, 1606, 1564, 1543, 1456, 1438, 1422, 1404, 1358, 1306, 1293, 1250, 1153, 1119; ¹H NMR (400 MHz, CDCl₃): δ 7.84 (d, 2H, J=16.0 Hz, CH), 7.62 (d, 2H, J=4.0 Hz, CH), 7.53 (d, 1H, J=2.3 Hz OCH═CH), 7.30 (d, 1H, J=12.0 Hz, CH), 6.95 (d, J=4.0 Hz, 2H, CH), 6.89 (d, 1H, J=12.0 Hz, CH), 6.88 (d, 1H, J=4.0 Hz, OCH═CH), 4.09 (s, 3H, OMe), 4.04 (s, 3H, OMe), 3.87 (s, 3H, OMe); ¹³C NMR (100 MHz, CDCl₃): δ (ppm) 194.6 (C═O), 161.7, 153.2, 151.7, 150.6, 144.1 (OCH═CH), 143.8, 130.3, 127.8, 124.5, 114.5, 113.4, 112.9, 112.0, 105.1 (OCH═CH), 62.0, 61.0, 55.4; HR-ESIMS: m/z 355.1171 [M+H]⁺ calcd for C₂₀H₁₈O₆+H⁺ (354.1103).

Example 8: Synthesis of 3-(4,7-dimethoxy-6-hydroxybenzofuran-5-yl)-1-(2,6-dichlorophenyl)-3-oxopropene (10). Procedure of synthesis is similar to example number 1 (steps land 2) except the respective starting material 2,6-dichlorobenzaldehyde is used in step 2. Orange yellow crystals; HPLC: t_{R}=5.3 min (100% purity); yield: 97%; m.p. 298-299° C.; IR (CHCl₃): νₘₐₓ 3399, 3161, 3090, 2951, 2921, 2851, 1640, 1613, 1577, 1472, 1441, 1427, 1378, 1357, 1328, 1301, 1275, 1242, 1213, 1185, 1145; ¹H NMR (400 MHz, CDCl₃): δ (ppm) 7.98 (d, 2H, J=8.0 Hz, CH), 7.52 (d, 1H, J=2.3 Hz, OCH═CH), 7.40 (d, 2H, J=8.0 Hz, CH), 7.23 (d, 1H, J=8.0 Hz, CH), 6.88 (d, 1H, J=4.0 Hz OCH═CH), 4.09 (s, 3H, OMe), 4.07 (s, 3H, OMe); ¹³C NMR (100 MHz, CDCl₃): δ (ppm) 194.4 (C═O), 153.4, 152.2, 151.1, 143.9 (OCH═CH), 141.7, 136.3, 135.3, 134.7, 132.6, 129.9, 129.3, 128.8, 112.3, 111.5, 105.3 (OCH═CH), 61.9 (OMe), 61.1 (OMe); HR-ESIMS: m/z 393.0287 (M+H⁺) calcd for C₁₉H₁₄Cl₂O₅+H⁺ (393.0291).

Example 9: Synthesis of 3-(4,7-dimethoxy-6-hydroxybenzofuran-5-yl)-1-(5-bromo-2-ethoxyphenyl)-3-oxopropene (11). Procedure of synthesis is similar to example number 1 (steps land 2) except the respective starting material 5-bromo-2-ethoxybenzaldehyde is used in step 2. orange powder; HPLC: t_{R}=5.1 min (100% purity) yield: 95%; m.p. 260-261° C., IR (CHCl₃): νₘₐₓ 3399, 3161, 3090, 2951, 2921, 2851, 1640, 1613, 1577, 1472, 1441, 1427, 1378, 1357, 1328; ¹H NMR (400 MHz, CDCl₃): δ (ppm) 8.12 (d, 1H, J=16.0 Hz, CH), 7.90 (d, 1H, J=16.0 Hz, CH), 7.73 (d, 1H, J=4.0 Hz, CH), 7.53 (d, 1H, J=2.2 Hz, OCH═CH), 7.43 (dd, 1H, J=4.0 Hz, CH), 6.88 (d, 1H, J=2.2 Hz, OCH═CH), 6.82 (d, 1H, J=8.0 Hz, CH), 4.13 (m, 2H, CH₂), 4.09 (s, 3H, OMe), 4.04 (s, 3H, OMe), 1.50 (t, 3H, J=7.0 Hz, Me); ¹³C NMR (100 MHz, CDCl₃): δ ppm 194.7 (C═O), 157.1, 157.0, 153.2, 151.9, 150.8, 144.2 (OCH═CH), 137.2, 134.0, 130.9, 128.1, 126.1, 113.9, 112.8, 112.8, 111.9, 106.2 (OCH═CH), 64.4, 62.0, 61.0, 14.7; HR-ESIMS: m/z 447.0433 [M+H]⁺ calcd for C₂₁H₁₉BrO₆+H⁺ (447.0437).

Example 10: Synthesis of 3-(4,7-dimethoxy-6-hydroxybenzofuran-5-yl)-1-(2-chlorophenyl)-3-oxopropene (12). Procedure of synthesis is similar to example number 1 (steps land 2) except the respective starting material 2-chlorobenzaldehyde is used in step 2. yellow powder; HPLC: t_{R}=5.1 min (100% purity); yield: 95%; m.p. 160-161° C. IR (CHCl₃): Vₘₐₓ 3435, 2922, 2851, 2650, 2342, 1693, 1628, 1591, 1571, 1469, 1439, 1408, 1363, 1316; ¹H NMR (400 MHz, CDCl₃): δ (ppm) 8.23 (d, J=16.0 Hz, 1H, CH), 7.86 (d, J=8.0 Hz, 1H, CH), 7.74 (d, 1H, J=2.2 Hz CH), 7.53 (d, 1H, J=2.3 Hz, OCH═CH), 7.33 (dd, J=2.3 Hz, J=4.0 Hz, 2H, CH), 6.88 (d, 1H, J=2.3 Hz, OCH═CH), 4.09 (s, 3H, OMe), 4.05 (s, 3H, OMe); ¹³C NMR (100 MHz, CDCl₃): δ (ppm) 194.4 (C═O), 153.1, 152.1, 150.7, 144.3 (OCH═CH), 141.1, 137.3, 133.1, 130.9, 130.5, 129.5, 128.3, 127.2, 123.1, 112.7, 111.7, 105.2, 62.0, 61.0 HR-ESIMS: m/z 359.0680 [M+H]⁺ calcd for C₁₉H₁₅ClO₅+H⁺ (359.0680).

Example 11: Synthesis of 3-(4,7-dimethoxy-6-hydroxybenzofuran-5-yl)-1-(2,3-dimethoxyphenyl)-3-oxopropene (13). Procedure of synthesis is similar to example number 1 (steps 1 and 2) except the respective starting material 2,3-dimethoxybenzaldehyde is used in step 2. Red solid; HPLC: t_{R}=4.7 min (100% purity); yield: 97%; m.p. 260-262° C.; IR (CHCl₃): Vₘₐₓ 3400, 2923, 2851, 1627, 1561, 1511, 1463, 1439, 1383, 1360, 1301, 1264, 1064, 1022; ¹H NMR (400 MHz, CDCl₃): δ (ppm) 7.83 (d, 2H, J=16.0 Hz, CH), 7.53 (d, 1H, J=4.0 Hz, OCH═CH), 7.25 (d, 2H, J=2.2 Hz, CH), 7.16 (d, 1H, J=4.0 Hz, CH), 6.91 (d, 1H, J=12.0 Hz, CH), 6.87 (d, 1H, J=4.0 Hz, OCH═CH), 4.09 (s, 3H, OMe), 4.03 (s, 3H, OMe), 3.95 (s, 3H, OMe), 3.93 (s, 3H, OMe); ¹³C NMR (100 MHz, CDCl₃): δ (ppm) 195.6 (C═O), 163.8, 161.1, 154.0, 152.2, 151.3, 144.7, 140.3, 131.4, 130.2, 125.3, 125.2, 118.0, 113.6, 112.9, 106.2, 105.8, 62.7, 61.7, 56.2, 56.2; HR-ESIMS: m/z 385.1278 [M+H]⁺ calcd for C₂₁H₂₀O₇+H⁺ (385.1281).

Example 12: Synthesis of 3-(4, 7-dimethoxy-6-hydroxybenzofuran-5-yl)-1-(2,4-dimethoxyphenyl)-3-oxopropene (14). Procedure of synthesis is similar to example number 1 (steps 1 and 2) except the respective starting material 3,4-dimethoxybenzaldehyde is used in step 2. colorless oil; HPLC: t_{R}=4.7 min (100% purity); yield: 95%; IR (CHCl₃): Vₘₐₓ 3400, 2923, 2851, 1627, 1561, 1511, 1463, 1439, 1383, 1360, 1301, 1264, 1064, 1022; ¹H NMR (400 MHz, CDCl₃): δ (ppm) 8.21 (d, 1H, J=16.0 Hz, CH), 7.93 (d, 1H, J=16.0 Hz, CH), 7.61 (d, 1H, J=8.0 Hz, CH), 7.51 (d, 1H, J=2.2 Hz, OCH═CH), 6.87 (s, 1H, CH), 6.53 (d, 1H, J=4.0 Hz, OCH═CH), 6.48 (s, 1H, CH), 4.09 (s, 3H, OMe), 3.91 (s, 3H, OMe), 3.87 (s, 3H, OMe); ¹³C NMR (100 MHz, CDCl₃): δ (ppm) 194.9 (C═O), 163.2, 160.5, 153.3, 151.5, 150.6, 144.0, 139.6, 130.7, 129.4, 124.6, 117.3, 112.9, 112.2, 105.5, 105.2, 98.4, 62.0, 61.0, 55.5, 55.5; HR-ESIMS: m/z 385.1276 [M+H]⁺ calcd for C₂₁H₂₀O₇+H⁺ (385.1281).

Example 13: Synthesis of 3-(4, 7-dimethoxy-6-hydroxybenzofuran-5-yl)-1-(2,3,4,5-pentafluorophenyl)-3-oxopropene (15). Procedure of synthesis is similar to example number 1 (steps 1 and 2) except the respective starting material 2,3,4,5,6-pentafluorobenzaldehyde is used in step 2. white solid; HPLC: t_{R}=5.0 min (92% purity); yield: 90%; m.p. 300-301° C.; IR (CHCl₃): Vₘₐₓ 3400, 2924, 2853, 1726, 1656, 1500, 1462, 1385, 1280, 1209, 1151, 1053, 1021; ¹H NMR (400 MHz, CDCl₃): δ H NMR (CDCl₃) 400 MHz): δ (ppm) 8.12 (d, 1H, J=16.0 Hz, CH), 7.81 (d, 1H, J=16.0 Hz, CH), 7.53 (d, 1H, J=4.0 Hz, OCH═CH), 6.91 (d, 1H, J=4.0 Hz, OCH═CH), 4.17 (s, 3H, OMe), 4.11 (s, 3H, OMe); ¹³C NMR (100 MHz, CDCl₃): δ (ppm); 194.3 (C═O), 153.5, 152.2, 151.0, 144.9, 144.1 (OCH═CH), 133.0, 132.9, 129.3, 127.2, 112.1, 111.3, 108.6, 105.1 (OCH═CH), 61.3, 61.0.; HR-ESIMS: m/z 415.0603 calcd for C₁₉H₁₁F₅O₅+H⁺ (415.0599).

Example 14: Synthesis of 3-(4,7-dimethoxy-6-hydroxybenzofuran-5-yl)-1-phenyl)-3-oxopropene (16). Procedure of synthesis is similar to example number 1 (steps 1 and 2) except the respective starting material benzaldehyde is used in step 2. yellow orange solid; HPLC: t_{R}=4.7 min (99% purity); yield: 90%; m.p. 123-126° C.; IR (CHCl₃): νₘₐₓ 3860, 3791, 3697, 3436, 3060, 2930, 2850, 1630, 1606, 1559, 1494, 1446, 1360; ¹H NMR (400 MHz, CDCl₃): δ (ppm) 7.88 (d, 2H, J=2.2 Hz, CH), 7.64-7.67 (m, 2H), 7.53 (d, 1H, J=2.2 Hz, OCH═CH), 7.42-7.45 (m, 3H), 6.89 (d, OCH═CH), 4.09 (s, 3H, OMe), 4.05 (s, 3H, OMe); ¹³C NMR (100 MHz, CDCl₃): δ (ppm) 194.7 (C═O), 153.2 (C-9a), 151.9 (C-6), 150.7 (C-4), 144.1 (CH═CH), 143.5 (OCH═CH), 135.1, 130.4, 129.6, 129.0, 128.5, 127.0, 112.8, 111.9, 105.2 (OCH═CH), 62.0, 61.0; HR-ESIMS: m/z 325.1078 [M+H]⁺ calcd for C₁₉H₁₆O₅+H⁺ (325.1075).

Example 15: Synthesis of 3-(4,7-dimethoxy-6-hydroxybenzofuran-5-yl)-1-(3-bromo-4-methoxyphenyl)-3-oxopropene (17). Procedure of synthesis is similar to example number 1 (steps land 2) except the respective starting material 3-bromo-4-methoxybenzaldehyde is used in step 2. orange crystals; HPLC: t_{R}=5.4 min (100% purity); yield: 92%; IR (CHCl₃): νₘₐₓ 3454, 2927, 2866, 1730, 1654, 1590, 1464, 1386, 1365, 1326, 1312, 1279, 1102, 1084, 1048; cm⁻¹H NMR (400 MHz, CDCl₃): δ (ppm): 8.11 (d, 1H, J=16.0 Hz, CH), 7.86 (d, 1H, J=16.0 Hz CH), 7.73 (s, 1H, CH), 7.52 (s, 1H, CH), 6.88 (s, 1H, CH), 6.84 (d, 1H, J=8.0 Hz, CH), 4.09 (s, 3H, OMe), 4.06 (s, 3H, OMe), 3.91 (s, 3H, OMe); ¹³C NMR (100 MHz, CDCl₃): δ (ppm) 194.8 (C═O), 157.7, 153.2, 151.9, 150.7, 144.2, 137.1, 134.0, 134.0, 130.9, 128.3, 128.3, 126.1, 113.0, 112.7, 105.2, 62.0 (OMe), 61.0 (OMe), 55.8; HR-ESIMS: m/z 432.0300 [M+H]⁺ calcd for C₂₀H₁₇BrO₆+H⁺ (432.0281).

Example 16: Synthesis of 3-(4,7-dimethoxy-6-hydroxybenzofuran-5-yl)-1-(2,4,5-trimethoxyphenyl)-3-oxopropene (18). Procedure of synthesis is similar to example number 1 (steps 1 and 2) except the respective starting material 3,4,5-trimethoxybenzaldehyde is used in step 2. reddish orange crystals; HPLC: t_{R}=8.7 min (100% purity); yield: 90%; IR (CHCl₃): νₘₐₓ 3434, 2930, 2867, 1724, 1656, 1517, 1463, 1385, 1263, 1210, 1159, 1084, 1024; ¹H NMR (400 MHz, CDCl₃): 8.29 (d, J=16.0 Hz, 1H, CH), 7.92 (d, J=16.0 Hz, 1H, CH), 7.59 (s, 1H, OCH═CH), 7.22 (m, 1H, CH), 6.94 (s, OCH═CH, 1H), 6.60 (s, 1H, CH), 4.16-3.97 (m, 15H, OMe); ¹³C NMR: (100 MHz, CDCl₃): δ (ppm) 194.7, 154.8, 153.2, 152.7, 151.5, 150.5, 144.0, 143.3, 139.3, 129.6, 124.4, 115.7, 113.0, 112.2, 111.2, 105.1, 96.7, 62.0 (OMe), 61.0 (OMe), 56.5 (OMe), 56.3 (OMe), 56.1 (OMe); HR-ESIMS: m/z 415.1390 [M+H]⁺ calcd for C₂₂H₂₁O₈+H⁺ (415.1390).

Example 17: Synthesis of 3-(benzo[d][1,3]dioxol-5-yl)-1-(6-hydroxy-4,7-dimethoxybenzofuran-5-yl)prop-2-en-1-one (19). Procedure of synthesis is similar to example number 1 (steps 1 and 2) except the respective starting material piperonal is used in step 2. Orange crystals; HPLC: t_{R}=5.6 min (92% purity) yield: 92%; IR (CHCl₃): νₘₐₓ 3743, 3385, 3130, 2850, 1729, 1627, 1565, 1542, 1489, 1470, 1446, 1353, 1300, 1255; ¹H NMR (400 MHz, CDCl₃): δ (ppm 7.78 (d, 2H, J=16.0 Hz, CH), 7.53 (d, J=4.0 Hz, OCH═CH, 1H), 7.15 (m, 2H, CH), 6.88 (d, J=2.2 Hz, OCH═CH, 1H), 6.87 (d, 1H, J=8.0 Hz, CH), 6.04 (s, 2H, CH₂), 4.09 (s, 3H, OMe), 4.04 (s, 3H, OMe); ¹³C NMR (100 MHz, CDCl₃): δ (ppm) 194.4 (C═O), 153.2, 151.7, 150.6, 149.9, 148.4, 144.1, 143.8, 143.6, 129.6, 125.4, 124.9, 112.8, 111.9, 108.7, 106.6, 105.2, 101.6, 62.0 (OMe), 61.0 (OMe); HR-ESIMS: m/z 369.0968 [M+H]⁺ calcd for C₂₀H₁₇O₇+H⁺ (369.0954).

Example 18: Synthesis of 1-(6-hydroxy-4,7-dimethoxybenzofuran-5-yl)-3-(thiophen-3-yl)prop-2-en-1-one (20). Procedure of synthesis is similar to example number 1 (steps 1 and 2) except the respective starting material thiophen-3-carboxaldehyde is used in step 2. Orange crystals; HPLC: t_{R}=3.9 min (85% purity); yield: 92%; IR (CHCl₃): νₘₐₓ 3584, 3136, 2922, 2850, 1626, 1586, 1561, 1543, 1470, 1442, 1364, 1416, 1297, 1131 cm-1; ¹H NMR (400 MHz, CDCl₃): δ (ppm) 7.89 (d, J=16.0 Hz, 1H, CH), 7.73 (d, J=16.0 Hz, CH), 7.62 (s, CH, 1H), 7.52 (s, 1H, CH), 7.40 (d, J=8.0 Hz, 2H, CH), 6.87 (s, CH, 1H), 4.09 (s, 3H, OMe), 4.03 (s, 3H, OMe); ¹³C NMR (100 MHz, CDCl₃): δ (ppm) 194.8 (C═O), 153.1, 151.8, 150.6, 144.1, 138.5, 137.2, 129.6, 129.1, 127.0, 126.6, 125.3, 112.8, 111.9, 105.1, 62.0 (OMe), 61.0 (OMe); HR-ESIMS: m/z 331.0619 [M+H]⁺ calcd for C₁₇H₁₅O₅S+H⁺ (331.0634).

Example 19: Synthesis of 3-(2,4-dichlorophenyl)-1-(6-hydroxy-4,7-dimethoxybenzofuran-5-yl)prop-2-en-1-one (21). Procedure of synthesis is similar to example number 1 (steps 1 and 2) except the respective starting material 2,4-dichlorobenzaldehyde is used in step 2. orange crystals; HPLC: t_{R}=5.0 min (95% purity) yield: 92%; IR (CHCl₃): νₘₐₓ 3399, 3161, 3090, 2951, 2921, 2851, 1640, 1613, 1577, 1472, 1441, 1427, 1378, 1357, 1328, 1301, 1275, 1242, 1213, 1185, 1145; ¹H NMR (400 MHz, CDCl₃): δ (ppm) 8.14 (d, J=16.0 Hz, 1H, CH), 7.83 (d, J=16.0 Hz, 1H, CH), 7.67 (d, J=12.0 Hz, 1H, CH), 7.53 (d, 1H, J=4.0 Hz, OCH═CH), 7.48 (d, J=3.0 Hz 1H, CH), 7.31 (m, 1H, CH), 6.88 (d, 1H, J=2.2 Hz, OCH═CH), 4.09 (s, 3H, OMe), 4.03 (s, 3H, OMe); ¹³C NMR (100 MHz, CDCl₃): δ (ppm) 194.2 (C═O), 153.2, 152.1, 150.7, 144.3, 137.5, 136.3, 136.1, 132.0, 130.1, 129.8, 129.5, 128.4, 127.6, 112.6, 111.7, 105.2, 61.9, 61.0; HR-ESIMS: m/z 393.0275 [M+H]⁺ calcd for C₁₉H₁₄C₂O₅+H+(393.0291).

Example 20: Synthesis of 1-(6-hydroxy-4,7-dimethoxybenzofuran-5-yl)-3-(naphthalen-2-yl)prop-2-en-1-one (22). Procedure of synthesis is similar to example number 1 (steps 1 and 2) except the respective starting material napthalen-2-benzaldehyde is used in step 2. Yellow powder; yield: 65%; HPLC: t_{R}=7.1 min (99% purity); IR (CHCl₃): νₘₐₓ 3584, 3136, 2922, 2850, 1626, 1586, 1561, 1543, 1470, 1442, 1379, 1297, 1149 cm-1; ¹H NMR (400 MHz, CDCl₃): δ (ppm) 8.03 (m, 3H, CH), 7.86 (m, 4H, CH), 7.54 (d, 1H, J=2.2 Hz, OCH═CH), 7.53 (m, 3H, CH), 6.90 (d, 1H, J=2.2 Hz, OCH═CH), 4.10 (s, 3H, OMe), 4.07 (s, 3H, OMe); ¹³C NMR (100 MHz, CDCl₃): δ (ppm) 194.6 (C═O), 153.5, 153.2, 150.7, 144.2, 143.7, 134.3, 133.4, 132.7, 130.7, 128.7, 128.6, 127.8, 127.4, 127.1, 126.8, 123.7, 112.9, 112.0, 110.6, 105.2, 62.1, 60.5; HR-ESIMS: m/z 375.1194 [M+H]⁺ calcd for C₂₃H₁₈O₅+H⁺ (375.1127).

Example 21: Synthesis of 3-(2-fluorophenyl)-1-(6-hydroxy-4,7-dimethoxybenzofuran-5-yl)prop-2-en-1-one (23). Procedure of synthesis is similar to example number 1 (steps 1 and 2) except the respective starting material 2-flurobenzaldehyde is used in step 2. Yellow powder; HPLC: t_{R}=5.0 min (95% purity); yield: 92%; IR (CHCl₃): νₘₐₓ 3400, 2922, 2851, 1628, 1601, 1556, 1544, 1510, 1461, 1443, 1413, 1360, 1297, 1299, 1266, 1151 cm⁻¹; ¹H NMR (400 MHz, CDCl₃): δ (ppm) 7.96 (q, 2H, J=15.8 Hz, CH), 7.64 (t, 1H, J=7.1 Hz, CH), 7.53 (d, 1H, J=2.2 Hz, OCH═CH), 7.38 (m, 1H, CH), 7.17 (m, 2H, CH), 6.89 (d, J=4.0 Hz, OCH═CH, 1H); ¹³C NMR (100 MHz, CDCl₃): δ (ppm) 194.8 (C═O), 162.8, 160.7, 153.3, 152.1, 150.9, 144.1, 136.0, 131.8, 129.8, 129.6, 124.5, 123.3, 116.4, 112.5, 111.7, 105.3, 61.8, 61.0; HR-ESIMS: m/z 432.0300 [M+H]⁺ calcd for C₂₀H₁₇BrO₆+H⁺ (432.0281).

Example 22: Synthesis of 3-(anthracen-2-yl)-1-(6-hydroxy-4,7-dimethoxybenzofuran-5-yl)prop-2-en-1-one (24). Procedure of synthesis is similar to example number 1 (steps 1 and 2) except the respective starting material 9-anthraldehyde is used in step 2. Yellow powder; HPLC: t_{R}=7.4 min (100% purity); yield: 92%; IR (CHCl₃): νₘₐₓ 3419, 2920, 2850, 2103, 1632, 1569, 1442, 1408, 1359, 1215, 1146, 1065 cm⁻¹; ¹H NMR (400 MHz, CDCl₃): δ (ppm) 8.82 (d, 1H, J=15.8 Hz, CH), 8.49 (s, 1H, CH), 8.40 (d, J=8.4 Hz, 2H, CH), 8.05 (d, J=7.4 Hz, 2H, CH), 7.76 (d, 1H, J=15.8 Hz, CH), 7.53 (m, 5H, CH), 6.87 (d, 1H, J=2.2 Hz, OCH═CH), 4.11 (s, 3H, OMe), 4.07 (s, 3H, OMe); ¹³C NMR (100 MHz, CDCl₃): δ (ppm) 194.3 (C═O), 153.3, 152.3, 150.9, 144.0, 140.2, 135.9, 131.3, 130.3, 129.7, 129.2, 128.98, 128.95, 128.47, 128.44, 128.3, 126.35, 126.33, 125.46, 126.44, 111.7, 111.5, 105.5, 61.4, 61.1; HR-ESIMS: m/z 425.1374 [M+H]⁺ calcd for C₂₇H₂₀O₅+H⁺ (425.1383).

Example 23. In-vitro CYP450 1A1 enzyme inhibition in Saccharosomes™: The screening method utilizes 384-well microplates to rapidly ascertain relative percentage inhibition of CYP1A1 by a library of compounds. Each reaction was performed in black, clear-bottomed 384-well microplates. A reaction volume of 50 μl comprised of 0.5 pmol of the cytochrome P450 CYP1A1 (Saccharosomes), 5 μM of ethoxyresorufin substrate (which contributes 0.05% DMSO to the well), 10 μM of potential inhibitor test article (which contributes 0.5% DMSO to the well), A P450 reductase NADPH regenerating system (1.3 mM NADP+, 3.3 mM glucose-6-0.02 units phosphate and glucose-6-Phosphate dehydrogenase), potassium phosphate buffer (final well concentration 100 mM, pH 7.4) and water. Very small quantities of magnesium chloride and sodium citrate are added to the NADPH regenerating system, in line with standard published protocols. The potential inhibitor (test article) was pre-incubated with CYP1A1 of at least 20 minutes at 30° C. After this period, the remainder of the reagents required in the assay was added to initiate the process. The reaction mixture was incubated for another 20 minutes at 30° C. The reaction was stopped by adding an 80% acetonitrile, 20% 0.5 M Tris solution. The reactions were monitored using the Biotek Synergy HT plate reader by measuring the endpoint reaction at Excitation 530/(25 bandwidth) & Emission 590/(20 bandwidth) using a gain/sensitivity setting of 60. The mean of the quadruplicates of the negative control (solvent inactivated CYP1A1) was deducted from the mean of potential inhibitor (test article) samples. The percentage was then derived relative to the mean of the wells without inhibitor.

The preliminary screening results of furanochalcones 1-24 for inhibition of CYP1A (Saccharosomes™) at 10 μM are shown in Table 1. The parent natural product khellin (1) showed potent inhibition (88%) of CYP1A1 at 10 VM. Several derivatives also showed potent inhibition of CYP1A1. This includes derivatives 5, 6, 8, 16, 18, 20 and 21 which showed >80% inhibition at 10 VM. Particularly, the compound 8 displayed very promising inhibition of CYP1A1 (97%), which was comparable to the positive control alpha-naphthoflavone.

TABLE 1
Inhibition of CYP1A1 (Saccharosomes ™) by furanochalcones 1-24

| Compound | | (% inhibition of CYP1A1 in |
|---|---|---|
| code | Structure | Saccharosomes ™ at 10 μM) |
| 1 | 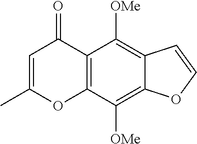 [SMILES: COc1c2occc2c(OC)c2c(=O)cc(C)oc12] | 88.3 |
| 2 | 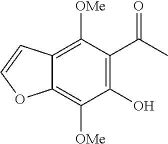 [SMILES: COc1c(C(C)=O)c(O)c(OC)c2occc12] | 47.5 |
| 3 | 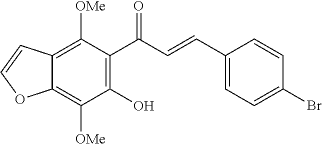 [SMILES: COc1c(C(=O)/C=C/c2ccc(Br)cc2)c(O)c(OC)c2occc12] | 26.8 |
| 4 | 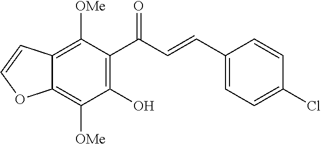 [SMILES: COc1c(C(=O)/C=C/c2ccc(Cl)cc2)c(O)c(OC)c2occc12] | 77.5 |
| 5 | 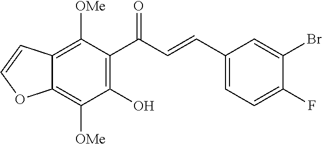 [SMILES: COc1c(C(=O)/C=C/c2ccc(F)c(Br)c2)c(O)c(OC)c2occc12] | 83.1 |
| 6 | 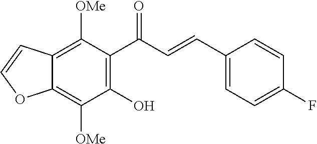 [SMILES: COc1c(C(=O)/C=C/c2ccc(F)cc2)c(O)c(OC)c2occc12] | 92.4 |
| 7 | 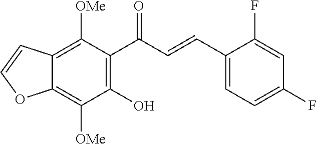 [SMILES: COc1c(C(=O)/C=C/c2ccc(F)cc2F)c(O)c(OC)c2occc12] | 23.9 |
| 8 | 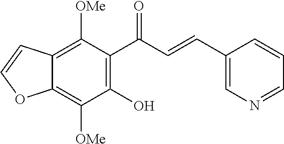 [SMILES: COc1c(C(=O)/C=C/c2cccnc2)c(O)c(OC)c2occc12] | 97.2 |
| 9 | 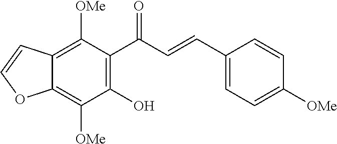 [SMILES: COc1ccc(/C=C/C(=O)c2c(O)c(OC)c3occc3c2OC)cc1] | 28.9 |
| 10 | 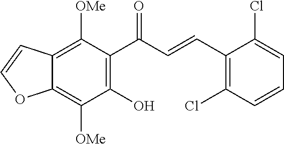 [SMILES: COc1c(C(=O)/C=C/c2c(Cl)cccc2Cl)c(O)c(OC)c2occc12] | 19.6 |
| 11 | 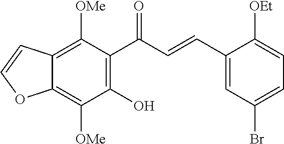 [SMILES: CCOc1ccc(Br)cc1/C=C/C(=O)c1c(O)c(OC)c2occc2c1OC] | 64.3 |
| 12 | 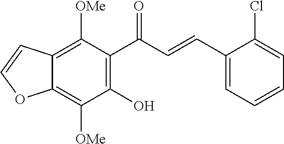 [SMILES: COc1c(C(=O)/C=C/c2ccccc2Cl)c(O)c(OC)c2occc12] | 23.6 |
| 13 | 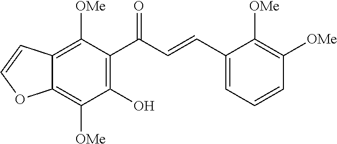 [SMILES: COc1cccc(/C=C/C(=O)c2c(O)c(OC)c3occc3c2OC)c1OC] | 15.2 |
| 14 | 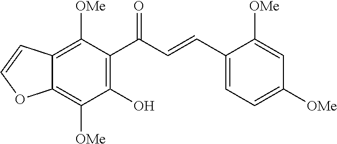 [SMILES: COc1ccc(/C=C/C(=O)c2c(O)c(OC)c3occc3c2OC)c(OC)c1] | 10.2 |
| 15 | 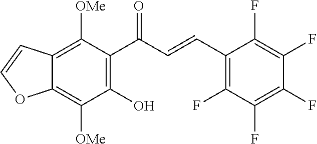 [SMILES: COc1c(C(=O)/C=C/c2c(F)c(F)c(F)c(F)c2F)c(O)c(OC)c2occc12] | 21.9 |
| 16 | 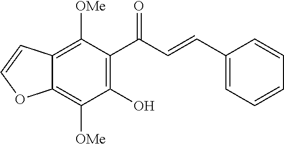 [SMILES: COc1c(C(=O)/C=C/c2ccccc2)c(O)c(OC)c2occc12] | 91.6 |
| 17 | 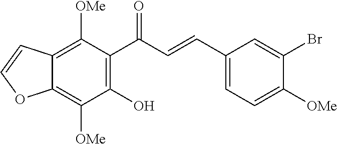 [SMILES: COc1ccc(/C=C/C(=O)c2c(O)c(OC)c3occc3c2OC)cc1Br] | 37.0 |
| 18 | 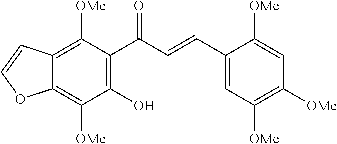 [SMILES: COc1cc(OC)c(OC)cc1/C=C/C(=O)c1c(O)c(OC)c2occc2c1OC] | 92.2 |
| 19 | 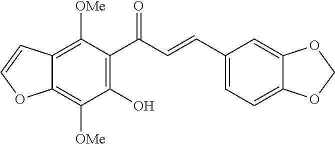 [SMILES: COc1c(C(=O)/C=C/c2ccc3c(c2)OCO3)c(O)c(OC)c2occc12] | 48.4 |
| 20 | 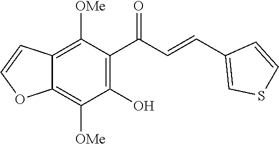 [SMILES: COc1c(C(=O)/C=C/c2ccsc2)c(O)c(OC)c2occc12] | 95.1 |
| 21 | 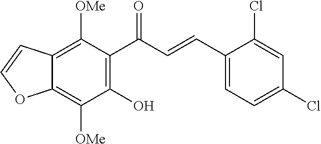 [SMILES: COc1c(C(=O)/C=C/c2ccc(Cl)cc2Cl)c(O)c(OC)c2occc12] | 80.2 |
| 22 | 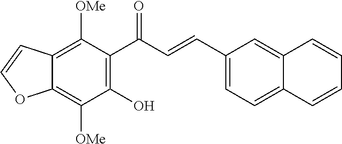 [SMILES: COc1c(C(=O)/C=C/c2ccc3ccccc3c2)c(O)c(OC)c2occc12] | 49.7 |
| 23 | 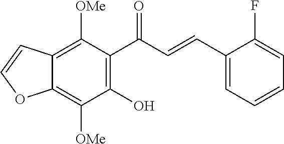 [SMILES: COc1c(C(=O)/C=C/c2ccccc2F)c(O)c(OC)c2occc12] | 41.1 |
| 24 | 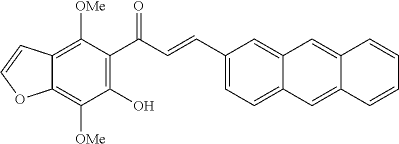 [SMILES: COc1c(C(=O)/C=C/c2ccc3cc4ccccc4cc3c2)c(O)c(OC)c2occc12] | 43.4 |
| Alpha- naphthoflavone | 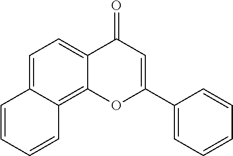 [SMILES: O=c1cc(-c2ccccc2)oc2c1ccc1ccccc12] | 97 |

Example 24. In-vitro CYP450 1B1 enzyme inhibition in Saccharosomes™: Regenerating system consists of: 5 μl Solution A (183 mg of NADP++183 mg of glucose-6-phosphate+654 μl of 1.0 M magnesium chloride solution+9.15 ml of sterile ultra-pure water)+1 μl Solution B (250 Units of glucose-6-phosphate dehydrogenase+6.25 ml of 5 mM sodium citrate; mixed in a tube and made up to 10 ml with sterile ultra-pure water)+39 μl 0.2 M Kpi (0.6 ml of 1.0M K2HPO4+9.4 ml of 1.0 M KH₂PO₄ were mixed and made up to 50 ml with sterile ultra-pure water)+5 μl potential inhibitory compound. Enzyme system consists of: 0.5 μl CYP1B1 (0.5 pmoles; CYP Design Ltd)+1.7 μl control protein (denatured proteins from yeast cells that do not contain recombinant CYP450 proteins)+5 μl 0.1 mM 7-ER (7-ethoxyresorufin substrate)+42.8 μl 0.1M Kpi (0.3 ml of 1.0 M K₂HPO₄+4.7 ml of 1.0 M KH₂PO₄ were mixed and made up to 50 ml with sterile ultra-pure water. The assay is performed using (a) sensitivity (Gain): 65/70/75 of the Biotek Synergy plate reader (this would differ from one instrument to the other) and (b) Filter: 530/590 nm that monitors fluorescence excitation/emission of resorufin, the metabolite of 7-ethoxyresorufin substrate (ER).

The preliminary screening results of furanochalcones 1-24 for inhibition of CYP1B1 (Saccharosomes™) at 10 SM are shown in Table 2. Amongst tested compounds, derivatives 8 and 20 showed >80% inhibition of CYP1B1 at 10 VM.

TABLE 2
Inhibition of CYP1B1 (Saccharosomes ™) by selected furanochalcones

| | | % inhibition of CYP1B1 in |
|---|---|---|
| Compound code | Structure | Saccharosomes ™ at 10 μM |
| 6 | 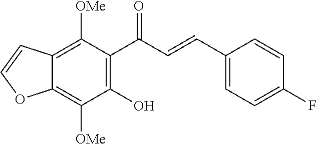 [SMILES: COc1c(C(=O)/C=C/c2ccc(F)cc2)c(O)c(OC)c2occc12] | 49.5 |
| 8 | 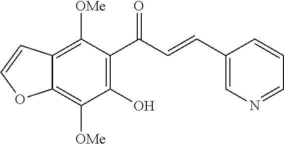 [SMILES: COc1c(C(=O)/C=C/c2cccnc2)c(O)c(OC)c2occc12] | 80.3 |
| 19 | 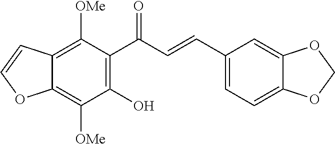 [SMILES: COc1c(C(=O)/C=C/c2ccc3c(c2)OCO3)c(O)c(OC)c2occc12] | 41.6 |
| 20 | 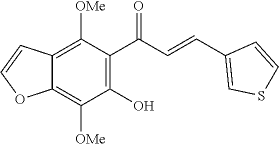 [SMILES: COc1c(C(=O)/C=C/c2ccsc2)c(O)c(OC)c2occc12] | 84.0 |
| 21 | 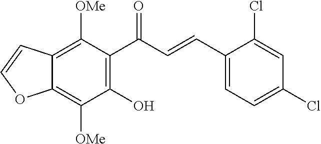 [SMILES: COc1c(C(=O)/C=C/c2ccc(Cl)cc2Cl)c(O)c(OC)c2occc12] | 51.4 |
| 22 | 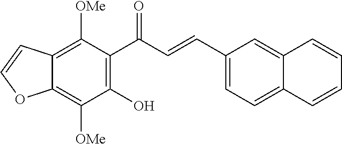 [SMILES: COc1c(C(=O)/C=C/c2ccc3ccccc3c2)c(O)c(OC)c2occc12] | 25.8 |
| 23 | 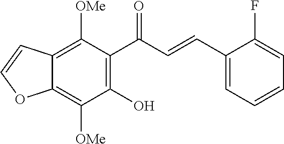 [SMILES: COc1c(C(=O)/C=C/c2ccccc2F)c(O)c(OC)c2occc12] | 34.2 |
| 24 | 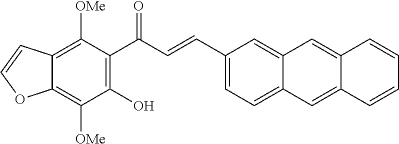 [SMILES: COc1c(C(=O)/C=C/c2ccc3cc4ccccc4cc3c2)c(O)c(OC)c2occc12] | 19.2 |
| Alpha- naphthoflavone | 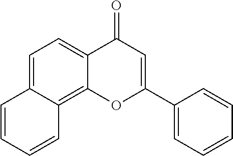 [SMILES: O=c1cc(-c2ccccc2)oc2c1ccc1ccccc12] | 98 |

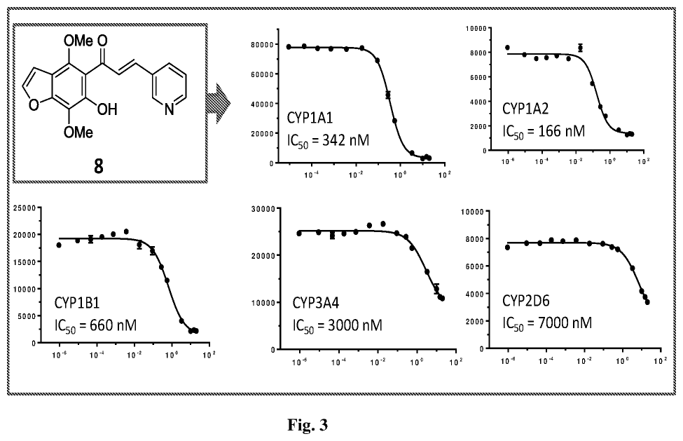

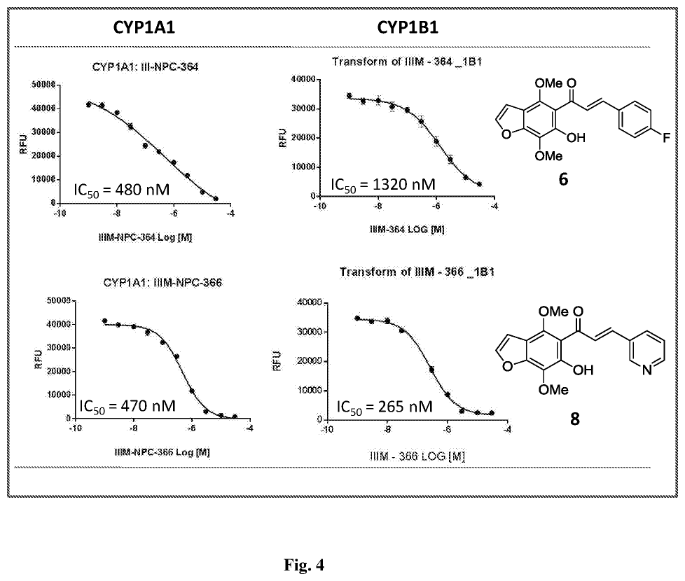

Example 25. IC₅₀ determination for best compound against CYP1A1, CYP1B1 and other CYPs in Saccharosomes. Compounds were serially diluted to six different concentrations with 10% DMSO in a Sero-Well white microplate. The experiment was performed in a similar way as described above in examples 23 and 24. Results of compound 8 are shown in Table 3. The dose-response curves of IC₅₀ determinations for selected CYP enzymes are shown in FIG. 3.

The pyridyl furanochalcone 8 showed potent inhibition of CYP1A1, CYP1A2 and CYP1B1 with IC₅₀ values of 342, 166 and 660 nM, respectively. Interestingly, this compound showed no inhibition of CYP2A6, 15% inhibition of CYP2B6, 24% inhibition of CYP2C8, and 7% inhibition of CYP2C19 at 20 μM. It showed 62, 63, and 84% inhibition of CYP2C9, CYP2C18 and CYP2D6 at 20 μM. This data is indicative of the fact that compound 8 is highly selective inhibitor of CYP1A1, CYP1A2 and CYP1B1, which are primarily involved in the cancer progression.

TABLE 3
IC₅₀ values of compound 8 against 12 CYPs in Saccharosomes^{a}

| Compound | CYP | IC₅₀ value |
|---|---|---|
| 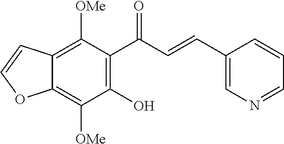 [SMILES: COc1c(C(=O)/C=C/c2cccnc2)c(O)c(OC)c2occc12] 8 | CYP1A1 CYP1A2 CYP1B1 CYP2A6 CYP2B6 CYP2C8 CYP2C9 CYP2C18 CYP2C19 CYP2D6 CYP2E1 | 342 nM 166 nM 660 nM No Inhibition at 20 μM. 15% Inhibition at 20 μM 24% Inhibition at 20 μM 62% Inhibition at 20 μM 63% Inhibition at 20 μM 7% Inhibition at 20 μM 7 μM No Inhibition at 10 μM |
| | CYP3A4 | 3 μM |
| 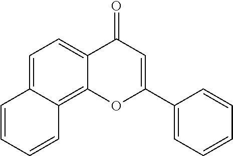 [SMILES: O=c1cc(-c2ccccc2)oc2c1ccc1ccccc12] Alpha-naphthoflavone | CYP1A1 | 90 nM |

^{a}The dose-response curves of IC₅₀ determinations for selected CYP enzymes are shown in FIG. 3.

Example 26. In-vitro CYP450 inhibition in HEK293 cells transfected with pcDNA3.1/CYP1A1 against 5 μM EROD. This assay was performed in a similar way as described above in examples 24 and 25. The HEK293 cells used here was procured from ‘European Collection of Authenticated Cell Cultures’ (catalog number. ECACC 85120602).

The results obtained in saccharosomes were then corroborated in live cells, for which the HEK290 cells transfected with pcDNA3.1/CYP1A1 was used. The preliminary screening was carried out at 10 μM. Results are shown in Table 4. Like in saccharosomes, the parent compound khellin (1) showed potent inhibition (81%) of CYP1A1 in live cells. Several compounds showed >80% inhibition of CYP1A1 in live cells at 10 μM; which includes compounds 2, 4, 5, 7, 8, 16 and 18.

TABLE 4
Inhibition of CYP1A1 in HEK293 cells

| Compound | | % inhibition of CYP1A1 in |
|---|---|---|
| code | Structure | HEK293 cells at 10 μM |
| 1 | 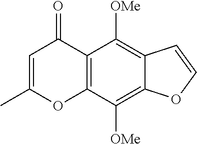 [SMILES: COc1c2occc2c(OC)c2c(=O)cc(C)oc12] | 81.3 |
| 2 | 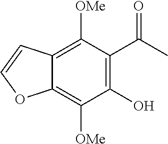 [SMILES: COc1c(C(C)=O)c(O)c(OC)c2occc12] | 81.1 |
| 3 | 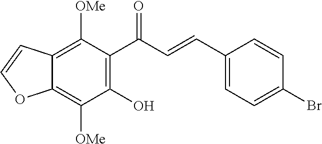 [SMILES: COc1c(C(=O)/C=C/c2ccc(Br)cc2)c(O)c(OC)c2occc12] | 57.7 |
| 4 | 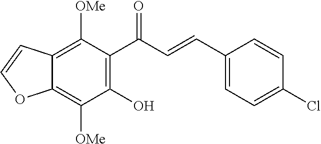 [SMILES: COc1c(C(=O)/C=C/c2ccc(Cl)cc2)c(O)c(OC)c2occc12] | 85.7 |
| 5 | 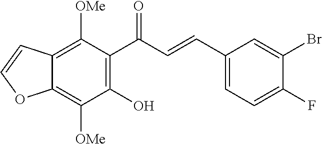 [SMILES: COc1c(C(=O)/C=C/c2ccc(F)c(Br)c2)c(O)c(OC)c2occc12] | 82.3 |
| 6 | 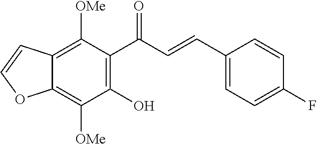 [SMILES: COc1c(C(=O)/C=C/c2ccc(F)cc2)c(O)c(OC)c2occc12] | 79.6 |
| 7 | 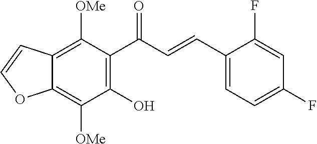 [SMILES: COc1c(C(=O)/C=C/c2ccc(F)cc2F)c(O)c(OC)c2occc12] | 85.4 |
| 8 | 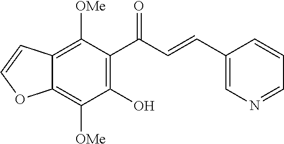 [SMILES: COc1c(C(=O)/C=C/c2cccnc2)c(O)c(OC)c2occc12] | 99.2 |
| 9 | 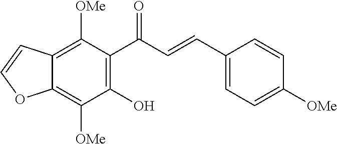 [SMILES: COc1ccc(/C=C/C(=O)c2c(O)c(OC)c3occc3c2OC)cc1] | 55.7 |
| 10 | 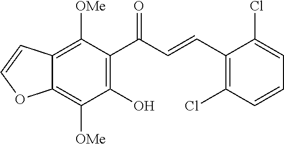 [SMILES: COc1c(C(=O)/C=C/c2c(Cl)cccc2Cl)c(O)c(OC)c2occc12] | 58.8 |
| 11 | 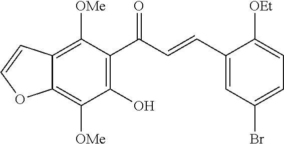 [SMILES: CCOc1ccc(Br)cc1/C=C/C(=O)c1c(O)c(OC)c2occc2c1OC] | 31.7 |
| 12 | 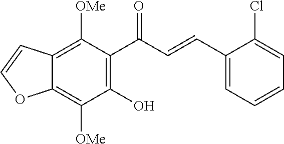 [SMILES: COc1c(C(=O)/C=C/c2ccccc2Cl)c(O)c(OC)c2occc12] | 19.5 |
| 13 | 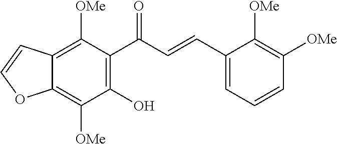 [SMILES: COc1cccc(/C=C/C(=O)c2c(O)c(OC)c3occc3c2OC)c1OC] | 21.6 |
| 14 | 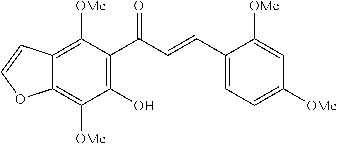 [SMILES: COc1ccc(/C=C/C(=O)c2c(O)c(OC)c3occc3c2OC)c(OC)c1] | 22.9 |
| 15 | 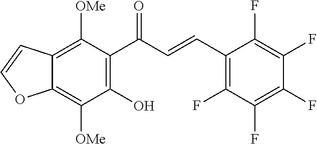 [SMILES: COc1c(C(=O)/C=C/c2c(F)c(F)c(F)c(F)c2F)c(O)c(OC)c2occc12] | 71.6 |
| 16 | 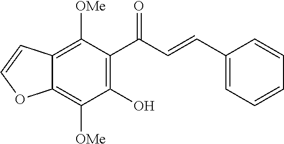 [SMILES: COc1c(C(=O)/C=C/c2ccccc2)c(O)c(OC)c2occc12] | 96.6 |
| 17 | 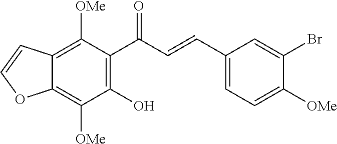 [SMILES: COc1ccc(/C=C/C(=O)c2c(O)c(OC)c3occc3c2OC)cc1Br] | 74.0 |
| 18 | 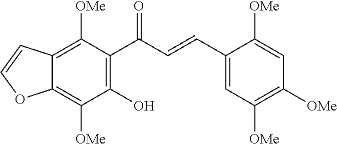 [SMILES: COc1cc(OC)c(OC)cc1/C=C/C(=O)c1c(O)c(OC)c2occc2c1OC] | 96.8 |
| 20 | 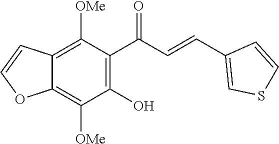 [SMILES: COc1c(C(=O)/C=C/c2ccsc2)c(O)c(OC)c2occc12] | 79.4 |
| 21 | 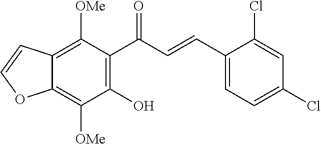 [SMILES: COc1c(C(=O)/C=C/c2ccc(Cl)cc2Cl)c(O)c(OC)c2occc12] | 71.4 |
| Alpha- naphthoflavone | 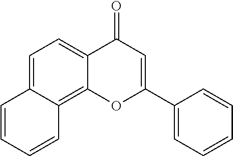 [SMILES: O=c1cc(-c2ccccc2)oc2c1ccc1ccccc12] | 30 |

Example 27. IC₅₀ determination of selected compounds against CYP1A1 and other CYP P450s in HEK293 cells transfected with pcDNA3.1/CYP1A1: The IC₅₀ values of selected compound 8 and 6 against CYP1A1 in Saccharosomes and in HEK293 cells transfected with pcDNA3.1/CYP1A1 was determined (Table 5). The dose-response curves of these IC₅₀ determinations are shown in FIG. 4.

The IC₅₀ values of best compounds 6 and 8 was then determined in live cells for CYP1A1 and CYP1B1 inhibition. Results are shown in Table 5. The furanochalcone 6 showed inhibition of CYP1A1 and CYP1B1 with IC₅₀ values of 480 and 1320 nM, respectively. Compound 8 showed IC₅₀ values of 470 and 265 nM against CYP1A1 and CYP1B1, respectively.

TABLE 5
IC₅₀ values of 6 against CYP1A1 and CYP1B1 in live cells^{a}

| | CYP1A1 | CYP1B1 |
|---|---|---|
| | IC₅₀ (in nM) | IC₅₀ (in nM) |
| Compound | (live human cells) | (live human cells) |
| 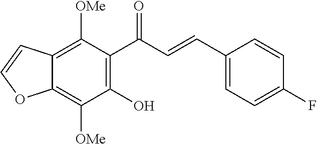 [SMILES: COc1c(C(=O)/C=C/c2ccc(F)cc2)c(O)c(OC)c2occc12] 6 | 480 | 1320 |
| 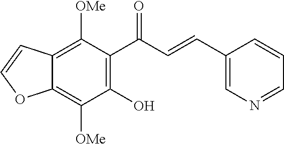 [SMILES: COc1c(C(=O)/C=C/c2cccnc2)c(O)c(OC)c2occc12] 8 | 470 | 265 |
| 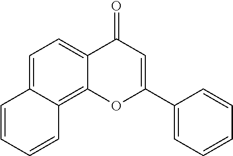 [SMILES: O=c1cc(-c2ccccc2)oc2c1ccc1ccccc12] Alpha-naphthoflavone | >10,000 | >10,000 |

^{a}The dose-response curves of these IC₅₀ determinations are shown in FIG. 4.

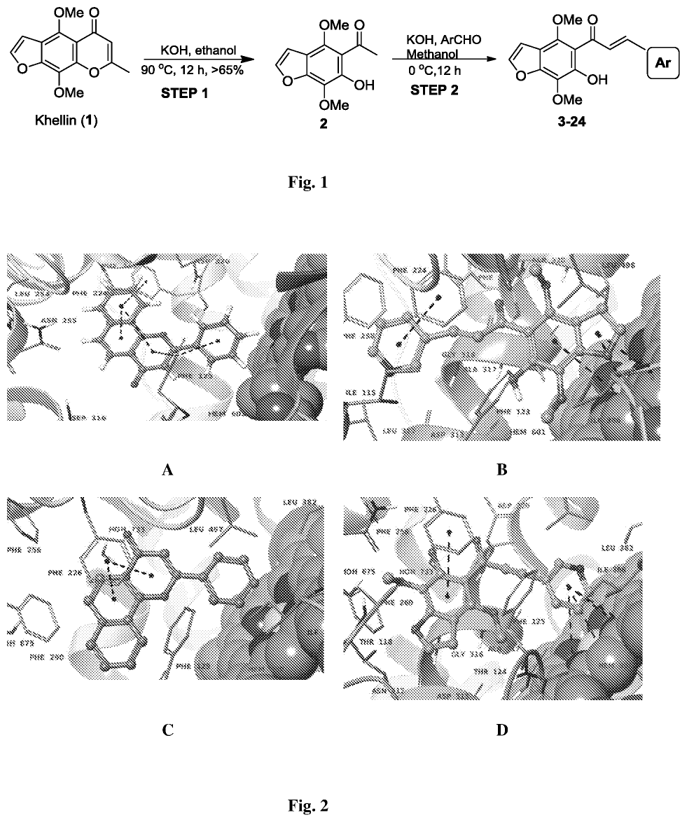

Example 28. Molecular modeling of compound 8 for CYP1A1 and CYP1A2 CYP1A1: The human CYP1A1 is an oxidoreductase enzyme belonging to the CYP1A sub-family. Its structure was published in 2013 by Walsh and co-workers (J. Biol. Chem. 2013, 288, 12932). The CYP1A1 crystal structure was retrieved from the Protein data bank (ID: 4I8V) and subjected to protein preparation wizard facility under default conditions implemented in Maestro v9.0 and Impact program v5.5 (Schrodinger, Inc., New York, N.Y., 2009). The prepared protein was further utilized to construct grid file by selecting alpha-naphthoflavone as centroid of grid box. The crystal structure of flavonoid α-naphthoflavone (ANF) was also retrieved from the Protein data bank, the ANF ligand being extracted from prepared enzyme-ligand complex. The rest of the chemical structures were sketched, minimized and docked using GLIDE XP. The ligand-protein complexes were minimized using macromodel, and the free energy (ΔG) of binding was calculated using Prime MMGB/SA function. Docked complex of the alpha-naphthoflavone, and compound with CYP1A1 is depicted in FIG. 2. Molecular docking of the claimed compound 8 display hydrophobic π-π interactions with the Phe224 and the highly hydrophobic Protoporphyrin IX containing FE complex.

CYP1A2: The human CYP1A2 is another oxidoreductase enzyme which belongs to the CYP1A sub-family. Its structure was solved in 2007 by Sansen and co-workers (J. Biol. Chem. 2007, 282, 14348). The CYP1A2 crystal structure was retrieved from Protein data bank (ID: 2HI4) and subjected to protein preparation wizard facility under default conditions implemented in Maestro v9.0 and Impact program v5.5 (Schrodinger, Inc., New York, N.Y., 2009). The prepared protein was further utilized to construct grid file by selecting alpha-naphthoflavone as centroid of grid box. The crystal structure of flavonoid alpha-naphthoflavone was also retrieved from the Protein data bank, the ANF ligand being extracted from prepared enzyme-ligand complex. The rest of the chemical structures were sketched, minimized and docked using GLIDE XP. The ligand-protein complexes were minimized using macromodel, and free energy (ΔG) of the binding was calculated using Prime MMGB/SA function. Docked complex of the alpha-naphthoflavone, and compound 8 with CYP1A2 is depicted in FIG. 3D-F. Molecular docking of the claimed compound 8 display hydrophobic π-π interactions with the Phe224 corresponding Phe226 residue of CYP1A2 and highly hydrophobic protoporphyrin IX containing FE complex.

### Advantages of the Invention

The main advantages of the present invention are:
1. Compounds of the invention show promising CYP1A1/CYP1A2/CYP1B1 inhibitory activity in-vitro yeast microsomes as well as in live human cells.
2. Compounds of the invention show selective inhibition of CYP1A1/CYP1A2/CYP1B1 enzymes over drug metabolizing cytochrome P450 enzymes CYP3A4 and CYP2D6.

1. A compound having formula A, 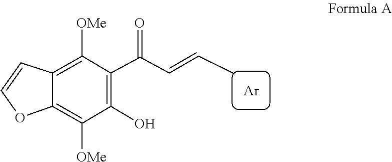
wherein, Ar is selected from the group consisting of 4-bromophenyl, 4-fluoro-3-bromo-phenyl, 2,4-difluorophenyl, 2,6-dichlorophenyl, 2-ethoxy-5-bromophenyl, 2,3-dimethoxyphenyl, 3-bromo-4-methoxyphenyl, 2,4,5-trimethoxyphenyl, thiophen-3-yl, 2,4-dichlorophenyl and anthracen-2-yl.

2. The compound as claimed in claim 1, wherein the compound having formula A is selected from the group consisting of:

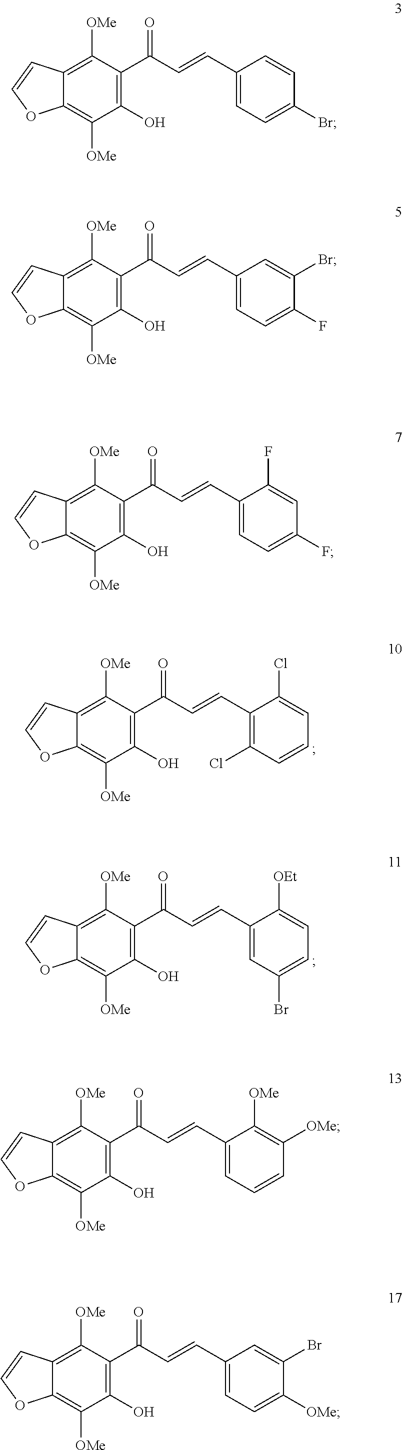

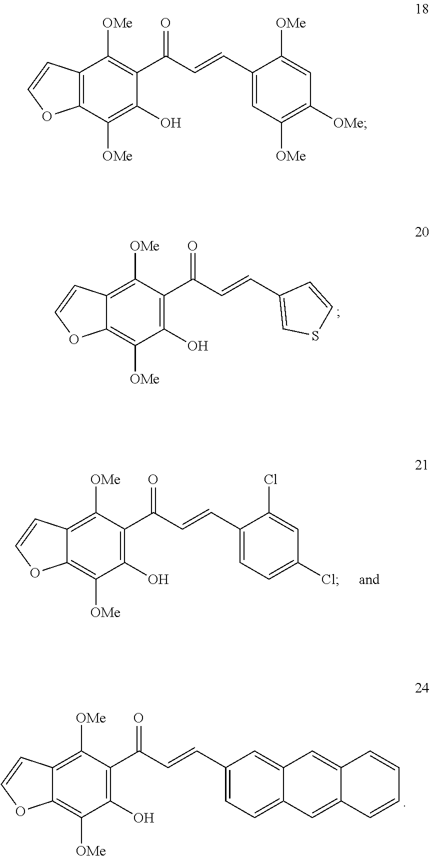 [SMILES: COc1cc(OC)c(OC)cc1/C=C/C(=O)c1c(O)c(OC)c2occc2c1OC ; COc1c(C(=O)/C=C/c2ccsc2)c(O)c(OC)c2occc12 ; COc1c(C(=O)/C=C/c2ccc(Cl)cc2Cl)c(O)c(OC)c2occc12 ; "and" ; COc1c(C(=O)/C=C/c2ccc3cc4ccccc4cc3c2)c(O)c(OC)c2occc12]

3. A method for preventing or treating cancer via inhibition of CYP1A1, CYP1A2, and CYP1B1 in a patient in need thereof, the method comprising administering a therapeutically effective amount of a compound having formula A, 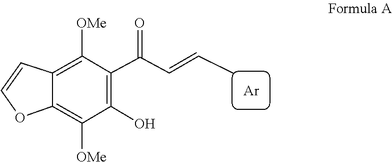
wherein, Ar is selected from the group consisting of 4-bromophenyl, 4-fluoro-3-bromo-phenyl, 2,4-difluorophenyl, 2,6-dichlorophenyl, 2-ethoxy-5-bromophenyl, 2,3-dimethoxyphenyl, 3-bromo-4-methoxyphenyl, 2,4,5-trimethoxyphenyl, thiophen-3-yl, 2,4-dichlorophenyl, anthracen-2-yl, 4-chlorophenyl, 4-fluorophenyl, pyridine-3-yl, 4-methoxyphenyl, 2-chlorophenyl, 2,4-dimethoxyphenyl, pentafluorophenyl, phenyl, 3,4-methylene-dioxy-phenyl, naphth-2-yl, and 2-fluorophenyl.

4. The method as claimed in claim 3, wherein the compound having Formula A is selected from the group consisting of:

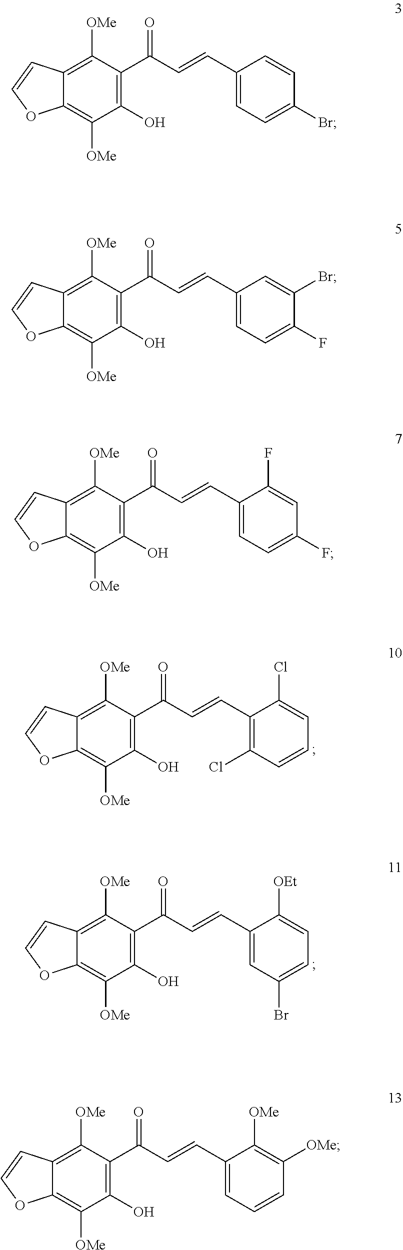 [SMILES: COc1c(C(=O)/C=C/c2ccc(Br)cc2)c(O)c(OC)c2occc12 ; COc1c(C(=O)/C=C/c2ccc(F)c(Br)c2)c(O)c(OC)c2occc12 ; COc1c(C(=O)/C=C/c2ccc(F)cc2F)c(O)c(OC)c2occc12 ; COc1c(C(=O)/C=C/c2c(Cl)cccc2Cl)c(O)c(OC)c2occc12 ; CCOc1ccc(Br)cc1/C=C/C(=O)c1c(O)c(OC)c2occc2c1OC ; COc1cccc(/C=C/C(=O)c2c(O)c(OC)c3occc3c2OC)c1OC]

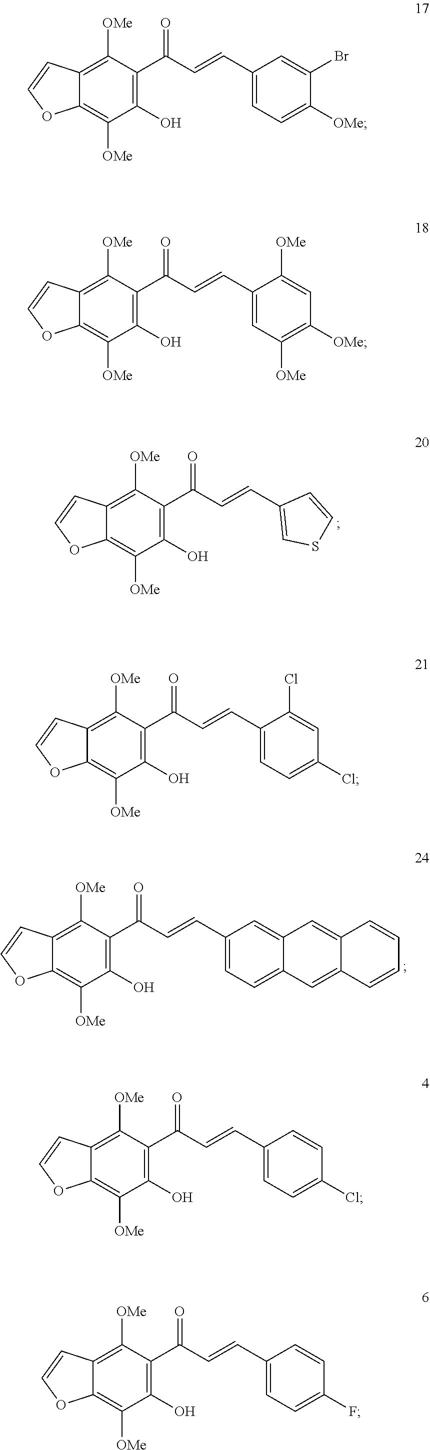

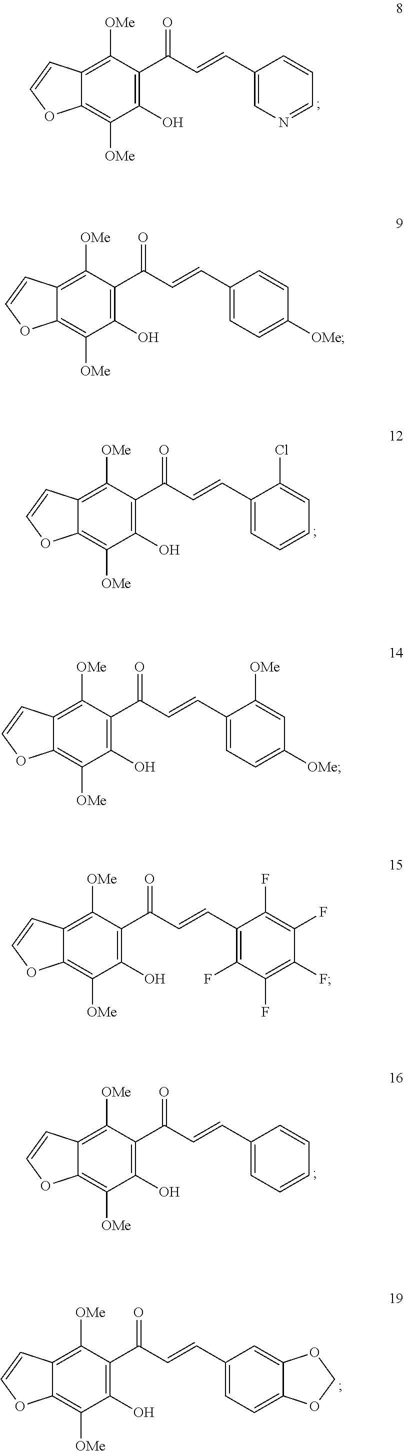

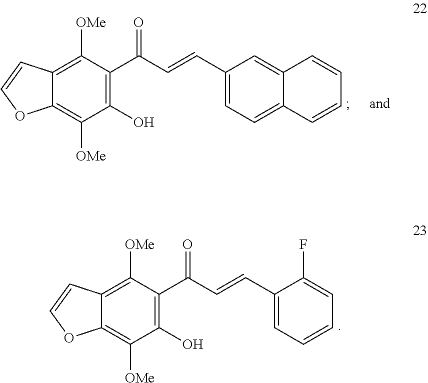 [SMILES: COc1c(C(=O)/C=C/c2ccc3ccccc3c2)c(O)c(OC)c2occc12 ; COc1c(C(=O)/C=C/c2ccccc2F)c(O)c(OC)c2occc12]

5. The method as claimed in claim 3, wherein the administering is effective to overcome chemo-resistance to cisplatin, docetaxel and paclitaxel through the inhibition of CYP1B1.

6. The method as claimed in claim 4, wherein IC₅₀ value of compound 8 is 342 and 470 nM against CYP1A1 in Sacchrosomes and in live cells.

7. A process for preparation of the compound having Formula A as claimed in claim 1, wherein the process comprises:
a. reacting khellin with an alkali hydroxide in an alcohol at reflux temperature ranging between 80-120° C. over a period in the range of 12-14 hours to form a reaction mixture, followed by concentrating the reaction mixture and extracting with an aqueous solvent selected from the group consisting of DCM: H₂O, chloroform: H₂O, and acetone: H₂O to obtain khellinone; and
b. reacting the khellinone obtained in (a) with an aldehyde in presence of a catalytic amount of an alkali selected from the group consisting of KOH and NaOH in an alcohol selected from the group consisting of methanol and ethanol at a temperature in the range of 0° C. to 1° C. over a period ranging between 12-14 hours to obtain the compound of Formula A.
c.

8. The process as claimed in claim 7, wherein the alkali hydroxide used in (a) is selected from the group consisting of sodium hydroxide and potassium hydroxide.

9. The process as claimed in claim 7, wherein the alcohol used in (a) is selected from the group consisting of ethanol and methanol.

10. The process as claimed in claim 7, wherein the aldehyde used in (b) is selected from the group consisting of 4-bromophenyl aldehyde, 4-fluoro-3-bromo-phenyl aldehyde, 2,4-difluorophenyl aldehyde, 2,6-dichlorophenyl aldehyde, 2-ethoxy-5-bromophenyl aldehyde, 2,3-dimethoxyphenyl aldehyde, 3-bromo-4-methoxyphenyl aldehyde, 2,4,5-trimethoxyphenyl aldehyde, thiophen-3-yl aldehyde, 2,4-dichlorophenyl aldehyde and anthracen-2-yl aldehyde, 4-chlorophenyl aldehyde, 4-fluorophenyl aldehyde, pyridine-3-yl aldehyde, 4-methoxyphenyl aldehyde, 2-chlorophenyl aldehyde, 2,4-dimethoxyphenyl aldehyde, pentafluorophenyl aldehyde, phenyl aldehyde, 3,4-methylene-dioxy-phenyl aldehyde, naphth-2-yl aldehyde, and 2-fluorophenyl aldehyde.

11. A pharmaceutical composition for the prevention or treatment of cancer, the pharmaceutical composition comprising an effective amount of at least one of the compound having structural Formulae A as claimed in claim 1 individually or in combination thereof, optionally, along with pharmaceutically acceptable excipients and/or diluents.

12. The pharmaceutical composition as claimed in claim 11, wherein the pharmaceutically acceptable excipients are saccharides selected from the group consisting of lactose, starch, and dextrose; stearates selected from the group consisting of stearic acid, magnesium stearate, polyvinylpyrrolidone, dicalcium phosphate dihydrate, eudragit polymers, celluloses, polyethylene glycol, polysorbate 80, sodium lauryl sulfate, magnesium oxide, and silicon dioxide; or carbonates selected from the group consisting of sodium carbonate, sodium bicarbonate, and talc.
